(19) United States
(12) Patent Application Publication
(10) Pub. No.: US 2015/0283208 A1
(43) Pub. Date: Oct. 8, 2015

(54) METHODS OF INHIBITING SURFACE ATTACHMENT OF MICROORGANISMS

(71) Applicants: Massachusetts Institute of Technology, Cambridge, MA (US); President and Fellows of Harvard College, Cambridge, MA (US)

(72) Inventors: Katharina Ribbeck, Cambridge, MA (US); Ronn S. Friedlander, Cambridge, MA (US); Kevin R. Foster, Cambridge, MA (US); Nicole Kavanaugh, Cambridge, MA (US); Marina Caldara, Cambridge, MA (US)

(21) Appl. No.: 14/677,051

(22) Filed: Apr. 2, 2015

Related U.S. Application Data
(63) Continuation of application No. PCT/US2013/032507, filed on Mar. 15, 2013.
(60) Provisional application No. 61/744,838, filed on Oct. 3, 2012.

(51) Int. Cl. A61K 38/17

(52) U.S. Cl. CPC A61K 38/1735

(57) ABSTRACT

The invention provides, inter alia, methods of inhibiting virulence of one or more microorganisms, and/or inhibiting one or more microorganisms from attaching to a surface, forming suspended aggregates or a combination thereof, by contacting the one or more microorganisms, the surface, or a combination thereof with a mucin, such as e.g., a purified, native, non-human mucin.

### RELATED APPLICATIONS

This application is a continuation of International Application No. PCT/US2013/032507, which designated the United States and was filed on Mar. 15, 2013, published in English, which claims the benefit of U.S. Provisional Application No. 61/744,838, filed on Oct. 3, 2012. The entire teachings of the above applications are incorporated herein by reference.

### BACKGROUND OF THE INVENTION

Many species of bacteria form surface-attached communities known as biofilms. Surrounded in secreted polymers, these bacterial aggregates are difficult to both prevent and eradicate, posing problems for medicine and industry.

### SUMMARY OF THE INVENTION

Shown herein is that natural systems provide a solution to prevent biofilms in the form of mucus, the hydrogel that coats the wet surfaces of vertebrates. Using defined in vitro assays, it was found that mucin biopolymers, the main functional constituents of mucus, acted as natural dispersants by promoting the motility of planktonic bacteria, and preventing their adhesion to underlying surfaces. The deletion of motility genes, however, allowed Pseudomonas aeruginosa to overcome the dispersive effects of mucus and form suspended antibiotic-resistant flocs, which mirror the immotile natural isolates found in the cystic fibrosis lung mucus. It was concluded that mucus is used by hosts to manipulate microbial behavior. Moreover, mucus offers new strategies that target bacterial virulence, and vexing engineering challenges, such as the design of anti-biofilm coatings for implants.

Accordingly, in one aspect, the invention is directed to a method of inhibiting one or more microorganisms from attaching to a surface, forming suspended aggregates or a combination thereof, comprising contacting the one or more microorganisms, the surface or a combination thereof with purified, native, non-human mucin.

In another aspect, the invention is directed to a method of inhibiting one or more microorganisms from forming a biofilm comprising contacting the one or more microorganisms with purified, native non-human mucin. The method can further comprise contacting a surface upon which the one or more microorganisms can form a biofilm, with purified, non-human mucin.

### BRIEF DESCRIPTION OF THE DRAWINGS

FIGS. 1A-1C: Mucins block P. aeruginosa attachment to surfaces. (1A) Images of coverglass surfaces at the indicated time points, depicting cell adhesion. Cells in PMM or PMM plus mucins were photographed at 2 s intervals at each time point. Images from these intervals were falsecolored red and green, respectively, and overlaid, allowing visualization of active cell motility or Brownian motion, versus firm adhesion. Scale bar is 10 μm. (1B) Number of wild-type cells firmly adherent to coverglass in PMM, or PMM supplemented with PEG, dextran or mucins after the indicated incubation periods. Error bars indicate s.e.m. of 8-11 different data points. (1C) PAO1 wild-type bacteria were grown in polypropylene tubes containing TB or TB plus 1% (w/v) PEG, dextran, or mucin. After 6 h, the relative amount of planktonic versus surface-attached cells was quantified using MTT staining Error bars represent the standard deviation.

FIGS. 2A-2F: Non-motile flagella mutants, but not their motile counterparts, form flocs in mucin environments. (2A) Boxplots depicting swimming velocities of P. aeruginosa in various conditions. Cells were grown in the media indicated, but swimming experiments were in 50%-strength media. Velocities were obtained from particle tracking analyses of 20-s swimming videos obtained at 20 frames per second. (2B) Floc formation of wild-type cells, flagella mutant (ΔflgE), a pili mutant (ΔpilB), and double flagella and pili mutant (ΔflgEΔpilB) in PMM with 1% mucins after 20 h of incubation. Images are of cells in suspension only. Scale bar is 20 μm. (2C, 2E, 2F) Boxplots quantifying floc size of wild type, flagella, pili and matrix and motility mutants for the strains indicated in μm2, after 20 h of growth in 1% mucin (unless otherwise indicated). For details on the quantification method see experimental procedures. For all boxplots, boxes extend from the 25th to the 75th percentile, the central line is the median, and whiskers extend to the data point nearest to 1.5 times the interquartile range above and below the box. Outliers are plotted as plus signs. (2D) Surface attached biofilm formation was quantified by crystal violet (CV): liquid cultures of the strains indicated were inoculated in 96-well plates at an OD600 of 0.01, and incubated for 7 h at 37° C. The biofilms that formed were quantified by staining with 0.1% CV as described previously [Friedman, L., and Kolter, R. (2004) J Bacteriol 186, 4457-65]. After staining, each plate was rinsed, and the remaining CV was destained with 33% acetic acid for 15 min. and measured using a plate reader (OD595). Data are presented as percent biofilm formation relative to wild type. The error bars represent standard deviation. See also FIGS. 5A-5J.

FIGS. 3A-3D: Flocs formed in mucin environments are exopolysaccharide-dependent. (3A) ΔflgE strains were grown for 20 h in PMM containing 1% (w/v) either PEG, dextran or industrially purified mucins (NBS Biologicals). Only in the presence of native mucins is floc formation observed. Scale bar is 20 μm. (3B) Liquid cultures of EPS secretion mutants and motility mutants were quantified by CV, as described in FIGS. 2A-2F. The experiments were performed in triplicate. The error bars represent the standard deviations. (3C) Boxplots of floc size of wild-type cells and the indicated motility and matrix mutants in PMM with 1% mucins after 20 h of incubation. Boxplots are drawn as described in FIGS. 2A-2F. See also FIGS. 6A-6D

FIGS. 4A-4C: Flocs grown in mucin environments are antibiotic resistant. (4A) Wild-type and ΔflgE cells were grown in liquid culture or in 1% mucin for 20 h and then exposed to colistin and ofloxacin. After 3 h of antibiotic exposure, the cells were plated to determine the number of surviving cells. (4B, 4C) Data from (4A) replotted as survival of antibiotic treated cells as a percentage of CFUs of untreated cells. Each trial was repeated at least 3 times. Error bars represent s.e.m. **, p<0.01; ***, p<0.001, comparing survival of ΔflgE to wild type in 1% mucins.

FIGS. 5A-5J: Motility is maintained in mucin environments; immotile cells form flocs. (5A) Time-lapse images of swimming P. aeruginosa in 50% PMM or 50% PMM+0.5% mucin. (5B) Time-lapse images of swimming E. coli in 50% M63 (upper) or 50% M63+0.5% mucin (lower). In (5A & 5B), images are at 0.2 s intervals and individual cell tracks are marked. Scale bars are 5 μm. (5C) Mean swimming velocities of E. coli in 50% M63 with or without 0.5% mucins. Velocities were obtained from particle tracking analyses of 20-s swimming videos obtained at 20 frames per second. ***, p<0.001 by two-tailed Student's t-test. Error bars indicate s.e.m. for n≧652 trajectories. (5D) Number of ΔflgE cells firmly adherent to coverglass in PMM, or PMM supplemented with PEG, dextran or mucins after the indicated incubation periods. Error bars indicate s.e.m. for 4-11 data points. (5E) The average duplication time of PAO1 in TB or TB plus mucins was determined over a time span of 6 h by CFU counting. Error bars represent standard deviation. (5F) Fluorescence micrographs depicting floc formation of wild-type PAO1 cells and flagella mutants (ΔflgE, ΔfliD, ΔflgK) in PMM with 1% mucins after 20 h of incubation. Images are of cells in suspension. Scale bar is 20 μm. (5G) Exponential phase cultures were added to PMM containing 1% mucins at a final concentration of 50-100 cells/W. The initial number of cells was determined by measuring the CFU. The culture was then incubated at 37° C. for 20 hrs. At the end of the incubation the culture was bead bashed and diluted with PBS to determine the final number of cells by CFU. Knowing the initial and final number of cells present in the mix, we could estimate the average duplication time of each strain. Each sample was inspected by microscopy to ensure that no aggregates were present. (5H) Floc formation by PA14 wild-type and mutant strains is quantified by projected area, indicated in μm2, after 20 h of growth in 1% mucin. Boxplots are drawn as described for FIGS. 2A-2F. (5I) Fluorescence micrographs depicting floc formation of wild-type PA14 cells and flagella/motility mutants (ΔflgK, ΔmotABΔmotCD) in 1% mucins after 20 h of incubation. Scale bar is 20 μm. (J) Surface attached biofilm formation was quantified by CV after 7 h of growth without mucins, as described for FIGS. 2A-2F. Error bars represent standard deviation.

FIGS. 6A-6D: Complementation of ΔflgE strains restores swimming motility and abolishes floc formation in mucin. The ΔflgE mutants were complemented using the plasmid pMQflgE (pMQ80 containing the flgE gene). As a control, pMQ80 plasmid without flgE was used (“empty”). (6A) Swimming motility of the genetically complemented ΔflgE mutant. The center of the agar plate is inoculated with bacteria. Only those cells that can swim will spread out from the center. (6B) Growth of the flgE complemented ΔflgE mutant in mucin. Scale bar is 50 μm. (6C) Swimming motility of the ΔflgEΔalgD and ΔflgEΔpsl double mutants. (6D) Growth of flgEcomplemented ΔflgE ΔalgD and ΔflgEΔpsl double mutants in mucin. Scale bar is 50 μm.

FIG. 7: Non-motile flagella mutants form clonal flocs in mucin environments. A GFPtagged ΔflgE mutant was mixed 1:1 with the un-tagged strain, and the mixture was inoculated in PMM containing 1% mucins. Images were taken after 8, 14 and 20 h. The top panels are visualized by bright field, the middle panels by fluorescence microscopy, and the bottom panels show overlays of the two channels. Scale bar is 20 μm.

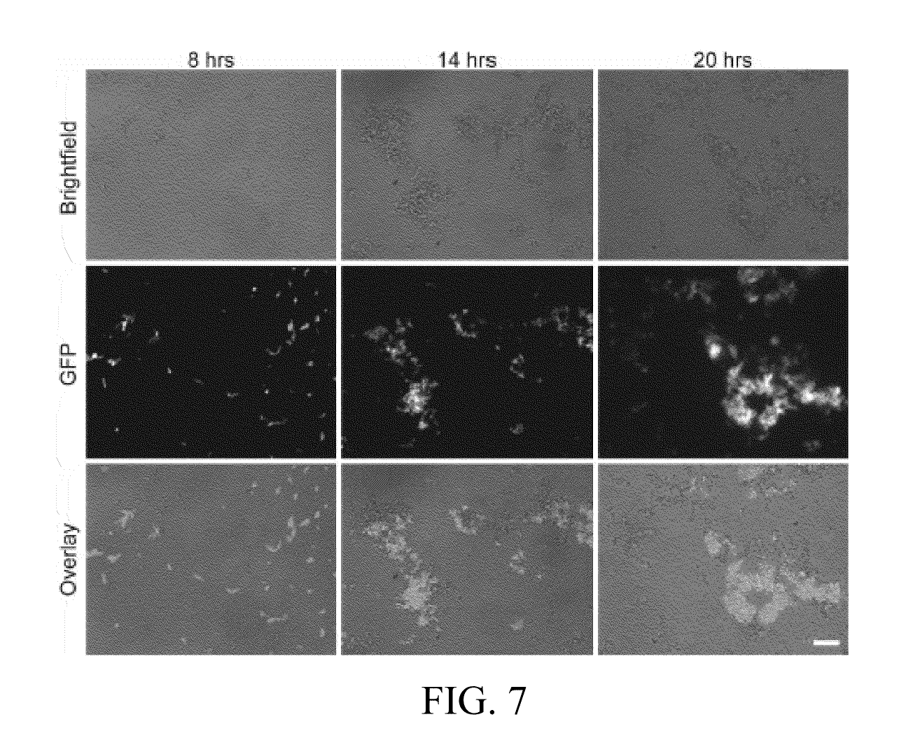

FIG. 8 is a schematic illustrating the effects of mucin on biofilms.

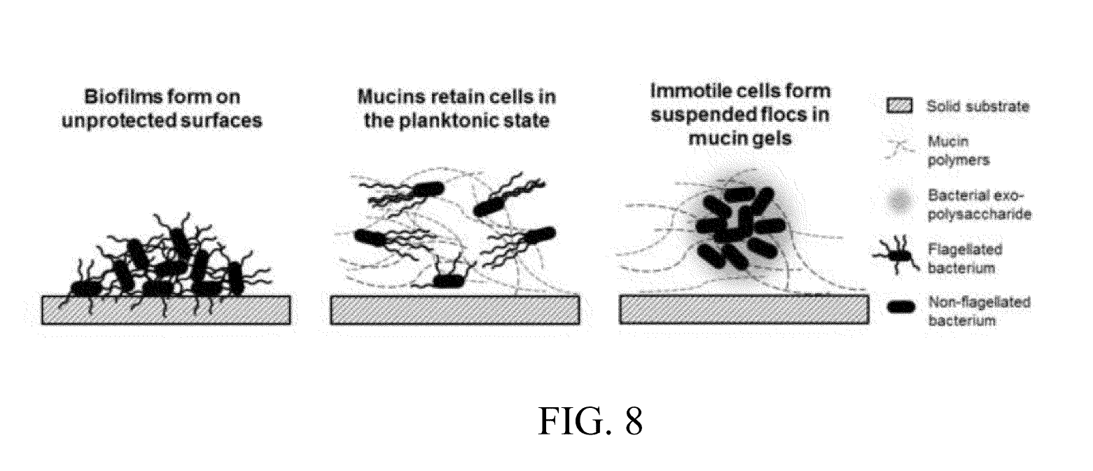

FIGS. 9A and 9B: Mucins affect the regulation of a number of genes implicated in virulence processes. The number of genes in different categories of virulence-associated genes that were upregulated (9A) or downregulated (9B) in the presence of mucin as determined by microarray analysis. The experiment was performed in two different media, namely RPMI and YPD+10% FBS. The graph depicts genes that are similarly regulated in both conditions.

FIGS. 10A and 10B: Mucins block attachment of C. albicans to polystyrene. (10A) Fluorescent microscopy images of polystyrene 96-well plates after incubation with C. albicans in different media conditions (RPMI alone, RPMI+0.5% methylcellulose, 0.5% industrially purified mucins or 0.5% native mucins). Time points were taken every 15 minutes by washing away non-adherent cells. The cells were stained with calcofluor white for visualization (10B) Quantification of attachment to the polystyrene plates. The colonized area of each frame was measured. The experiment was performed in triplicate with 5 pictures of each well taken at each time point (15 pictures total for each condition/timepoint). The error bars represent standard deviation.

FIGS. 11A and 11B: Mucins block attachment of C. albicans to human mucus-secreting intestinal cells. (11A) Fluorescence and phase contrast microscopy images of C. albicans MLR62 (constitutive GFP expressing strain) after 2 hours of incubation with HT29-MTX human mucus-secreting cells. The HT29-MTX cells were incubated with PBS as a control (+mucus) or with N-acetylcysteine to remove the adherent mucus layer (−mucus). (11B) Quantification of C. albicans attachment to HT29-MTX cells. Error bars represent standard deviation of 3 replicates.

FIGS. 12A-12E: Mucins suppress the transition from yeast form cells to invasive hyphae. Yeast form cells were incubated for 8 hours in RPMI (12A), 0.5% Methylcellulose (12B), 0.5% native mucins (12C) or 0.5% industrially purified mucins (12D). The strain used, HGFP3, expresses GFP only when cells are true hyphae. (12E) Quantitative PCR of hyphae-specific gene expression after growth in RPMI, 0.5% Methylcellulose, 0.5% native mucins, or 0.5% industrially purified mucin. RNA was extracted from biological triplicates and qPCR was performed in duplicate. Error bars represent standard deviation.

### DETAILED DESCRIPTION OF THE INVENTION

A description of example embodiments of the invention follows.

Biofilms, or surface attached communities of microbes, have gained interest due to their ability to colonize inert and biological surfaces [Donlan, R. M. (2001) Clin. Infect. Dis. 33, 1387-1392]. Once bacteria colonize a surface, they surround themselves in secreted polymers and firmly attach to the substrate [Petrova, O. E. and Sauer, K. (2012) J. Bacteriol. jb.asm.org/content/early/2012/02/27/JB.00003-12 [Accessed May 29, 2012]]. Humans play host to hundreds of trillions of microbes that live adjacent to our epithelia [Whitman, W. B. et al. PNAS 95, 6578-6583] and are typically able to prevent harmful colonization events. It appears that natural selection would necessitate the evolution of a biofilm-limiting material. Several such polymers, produced by microbes, have been described, whose putative function is to block competing biofilms [Bendaoud, M. et al. (2011). J. Bacteriol. 193, 3879-3886; Valle, J., et al. (2006) Proc. Natl. Acad. Sci. U.S.A. 103, 12558-12563; Kim, Y. et al. (2009) Biochem. Biophys. Res. Commun. 379, 324-329].

As shown herein, mucus, the hydrogel that coats all wet surfaces in the human body, regulates surface colonization by microbes. Mucus is largely perceived as a passive barrier that traps potentially deleterious particles or pathogens and is continuously shed. Indeed, mucus can sequester a diverse range of particles, including protons [Schade, C. et al. (1994) Gastroenterology 107, 180-188] and viruses [Lai, S. K. et al. (2009) J. Virol. 83, 11196-11200; Lai, S. K. et al. (2010) Proc. Natl. Acad. Sci. USA 107, 598-603; Lieleg, O. et al. (2012) www.ncbi.nlm.nih.gov/pubmed/22475261], thereby limiting their access to an underlying surface. Mucus can also prevent bacterial contact with the underlying epithelia. The digestive tract, for example, is lined by a firmly adherent mucus layer that is typically devoid of bacteria, followed by a second, loosely adherent layer that contains numerous bacteria [Atuma, C. et al. (2001) Am. J. Physiol. Gastrointest. Liver Physiol. 280, G922-929; Johansson, M. E. V. et al. (2008) Proc. Natl. Acad. Sci. U.S.A. 105, 15064-15069]. In addition, the mucus harbors immune factors, such as antibodies and defensive enzymes to aid in host defense [McGuckin, M. A. et al. Nat Rev Microbiol 9, 265-78]. However, as shown herein, the mucus gel itself acts as an active antimicrobial agent. For example, mucin glycoproteins, the major constituents of the mucus barrier, can block cell wall synthesis in Helicobacter pylori, thereby limiting cell growth [Kawakubo, M. et al. (2004) Science 305, 1003-1006]. As shown herein, the mucus barrier is a sophisticated bioactive material, and plays an instrumental role in managing bacterial surface colonization.

Accordingly, in one aspect, the invention is directed to methods of inhibiting virulence of one or more microorganisms, and/or inhibiting one or more microorganisms from attaching to a surface, forming suspended aggregates or a combination thereof, by contacting the one or more microorganisms, the surface or a combination thereof with a mucin, such as purified non-human mucin (e.g., purified, native, non-human mucin).

In another aspect, the invention is directed to a method of inhibiting one or more microorganisms from forming a biofilm comprising contacting the one or more microorganisms with purified, native non-human mucin. The method can further comprise contacting a surface upon which the one or more microorganisms can form a biofilm, with purified non-human mucin (e.g., purified, native, non-human mucin). As used herein a “biofilm” refers to a structured community of cells of a microorganism enclosed in a (e.g., self-produced) polymeric matrix that is adherent to a surface (e.g., an inert surface; a living surface).

“Mucin” and the like is a highly glycosylated protein capable of forming gels, generally comprising an amino and/or carboxy regions that are cysteine-rich and a central region enriched for serine and/or threonine residues and associated O-linked and/or N-linked oligosaccharides. Exemplary mucins include, for example, certain human mucins such as MUC1 (human GeneID No. 4582), MUC2 (human GeneID No. 4583), MUC5AC (human GeneID No. 4586), and MUC5B (human GeneID No. 727897). In certain embodiments, the mucin is a MUC5AC mucin (see, e.g. UniGene IDs 3881294, 1370646, 1774723, 1133368 and HomoloGene 130646), a MUC5B (see, e.g., HomoloGene 124413), a MUC6 (see, e.g., HomoloGene 18768), MUC2 (see, e.g., HomoloGene 130504, 131905, 132025, or 133451) or combinations thereof. In some particular embodiments, the mucin is a secreted mucin, such as MUC5AC, MUC5B, MUC6, and MUC2. In more particular embodiments, the mucin is a gastric mucin, such as MUC5AC, such as a porcine MUC5AC (see, e.g., UnigeneIDs 441382, 5878683; GeneID No. 100170143, and reference sequences AAC48526, AAD19833, and AAD19832). Other mucins suitable for use concordant with the invention include bovine submaxillary mucin (BSM, also known as MUC19; see e.g. GeneID No. 100140959; see HomoloGeneID 130967; see reference protein sequence XP_{—}003586112.1). A mucin-containing composition provided by the invention can be a mixture of one or more mucins (e.g., at least 2, 3, 4, 5, or more different mucins) and, optionally, may be made up of equal or unequal proportions of the different mucins—e.g., a particular mucin may, in certain embodiments, make up at least about 20, 30, 40, 50, 60, 65, 70, 75, 80, 85, 90, 95, 96, 97, 98, 99, or 100% of the mucins in the composition. Preferably, isolated or purified mucin comprises at least about 50%, 75%, 80%, 90%, 95%, 98% or 99% (on a molar basis) of all macromolecular species present.

Any of the individual mucin sequences described in the above annotations can be adapted for use in the invention, as well as variants thereof, e.g., sequences at least 50, 55, 60, 65, 70, 75, 80, 85, 90, 91, 92, 93, 94, 9, 96, 97, 98, 99, or 100% identical to a functional fragment thereof (e.g., comprising about 40, 50, 60, 70, 75, 80, 85, 90, 95, or 100% of the full length of the mature proteins) that is capable of forming a stable mucin surface. Functional variants will generally preserve the function of the conserved domains present in mucins, including one or more of a cyctine-rich domain, VWC (cl02515), GHB-like (cl00070), TIL (pfam01826) Mucin2_WxxW (pfam13330), VWD (cl02516), c8 (cl07383), and FA58C (cl12042) domains.

Mucins for use in the invention can be chemically or recombinantly (e.g. in CHO or COS cells) synthesized or isolated from a natural source, e.g., from non-human animals. The mucin can be obtained and purified using the methods described herein from any non-human mammal such as a non-human primate, a bovine, a porcine, a canine, a feline, an equine and the like. In a particular aspect, the non-human gastric mucin is porcine gastric mucin. Porcine gastric mucin can be isolated by the methods described in Celli, J., et al., Biomacrmolecules 2005, 6(3), 1329-1333, incorporated by reference in its entirety, preferably omitting the cesium density gradient centrifugation.

In one aspect, the mucin is non-human mucin. For example, the mucin can be a mucin from a non-human mammal. As used herein, the terms “mammal” and “mammalian” refer to any vertebrate animal, including monotremes, marsupials and placental, that suckle their young and either give birth to living young (eutharian or placental mammals) or are egg-laying (metatharian or nonplacental mammals). Examples of mammalian species that can be used to obtain mucin include primates (e.g., humans, monkeys, chimpanzees), rodents (e.g., rats, mice, guinea pigs), canines, felines, and ruminents (e.g., cows, porcine (pigs), horses).

The mucin for use in the methods described herein can be obtained from any mucus-containing region of the non-human mammal. Examples of mucins for use in the methods of the inventions include, for example, salivary mucin, nasal mucin, lung mucus (e.g., phlegm), cervical mucus, gastric mucin and the like. In a particular aspect, the mucin is purified, native porcine gastric mucin.

As used herein, a “native non-human” mucin refers to a non-human mucin that is purified in its native form. In one aspect, the mucin is purified to obtain an extract composed of one or more the gel-forming components of mucin (e.g., one or more of the gel-forming components found in the lungs and/or stomach of a non-human mammal). The gel forming units include MUC1, MUC2, MUC5AC, and MUC5B. Methods for purifying native, non-human mucin (e.g., native porcine gastric mucin) are described herein and are known in the art (e.g., Celli, J., et al. (2005) Biomacromolecules 6, 1329-33). In another aspect, the purified, native, non-human mucin can form viscoelastic hydrogels. In yet another aspect, the mucin is not a commercially available mucin.

As used herein, “isolated”, “purified” “substantially pure or purified” or “substantially isolated” refers to a mucin (e.g., gastric mucin) that is separated from the complex cellular milieu in which it naturally occurs, or chemical precursors or other chemicals when chemically synthesized. In some instances, the isolated or purified mucin comprises, consists essentially of, or consists of MUC5AC, MUC2, MUC5B MUC6 or combinations thereof. Preferably, isolated or purified mucin comprises at least about 50%, 55%, 60%, 65%, 70%, 75%, 80%, 85%, 90%, 95%, 98% or 99% (on a molar basis) of all macromolecular species present.

“Virulence” of a microorganism is a phenotypic state associated with, in some embodiments, infection that may (or does) harm the host, for example, the host's epithelial tissues. As an example, virulence can encompass one or more of modulation of virulence-associated genes (e.g. upregulating genes positively correlated with virulence and/or downregulating genes negatively correlated with virulence), and/or increasing invasive or virulent cell types, such as, for example filamentous forms, e.g., hyphae formation of yeast such as Candida albicans. In some embodiments, the methods provided by the invention prevent or inhibit virulence of one or more microorganisms.

In particular aspects, the mucin is in a solution. As will be appreciated by those of skill in the art, the mucin can be in a solution of a variety of solvents. Examples of such solvents include saline (e.g., phosphate buffered saline (PBS)), cell culture media (e.g., bacterial cell culture medium, mammalian cell culture medium), buffer solution and the like.

The concentration of the mucin used in the methods of the invention will vary and will depend on the desired use. In some aspects, the gastric mucin concentration is about 0.20% (w/v), 0.25% (w/v), 0.30% (w/v), 0.35% (w/v), 0.40% (w/v), 0.45% (w/v), 0.50% (w/v), 0.55% (w/v), 0.6% (w/v), 0.65% (w/v), 0.7% (w/v), 0.75% (w/v), 0.8% (w/v), 0.85% (w/v), 0.9% (w/v), 0.95% (w/v), 1% (w/v), 1.5% (w/v), 2.0% (w/v), 2.5% (w/v) in the solution. In a particular aspect, the concentration of mucin in solution is a physiological concentration of mucin (e.g., Kirkham, S et al. (2002) Biochem J, 361, 537-546).

In yet other aspect, the mucin has an acidic, basic or neutral pH (e.g., a pH about 2, 3, 4, 5, 6, 7, 8 9, 10, 11, 12, 13).

In further aspects, the mucin is in a solution that has a salt concentration of about 20 mM, 40 mM, 60 mM, 80 mM, 100 mM, 120 mM, 140 mM, 160 mM, 180 mM, 200 mM, 220 mM, 240 mM, 260 mM, 280 mM, 300 mM, 320 mM, 340 mM, 360 mM, 380 mM, 400 mM, 420 mM, 440 mM, 460 mM, 480 mM, 500 mM, 520 mM, 540 mM, 560 mM, 580 mM, 600 mM, 620 mM, 640 mM, 660 mM, 680 mM, 700 mM, 720 mM, 740 mM, 760 mM, 780 mM, 800 mM, 820 mM, 840 mM, 860 mM, 880 mM, 900 mM, 920 mM, 940 mM, 960 mM, 980 mM, or 1000 mM (1M).

Mucins for use in the present invention can, in some embodiments, be part of mucin/lectin multilayer films, as described in Internation Patent Application No. PCT/US2013/024978, filed Feb. 6, 2013, which is incorporated by reference in its entirety.

The methods described herein can be used to inhibit any microorganism capable of attaching to a surface, forming suspended aggregates, forming a biofilm or combinations thereof. In one aspect, the one or more microorganisms are planktonic microorganisms (e.g., motile; free swimming), sessile microorganisms (e.g., non-motile; attached to a surface) or a combination thereof. In another aspect, the one or more microorganisms are pathogenic or capable of pathogenicity. In yet other aspects, the one or more microorganisms include one or more bacteria, archaea, fungi (such as a yeast, such as Candida albicans) or a combination thereof.

As will be appreciated by those of skill in the art, the methods described herein can be used with any of the microorganisms classified using a variety of criteria. Examples of bacteria include bacteria classified by metabolism such as photoautotrophs (e.g., Cyanobacteria, Green sulfur bacteria, Chloroflexi, or Purple bacteria), photoheterotrophs, lithotrophs (e.g., chemolithoautotrophs, chemolithoheterotrophs such as Thermodesulfobacteria, Hydrogenophilaceae, or Nitrospirae), organotrophs (e.g., chemoorganoheterotrophs such as Bacillus, Clostridium or Enterobacteriaceae). Other examples of bacteria include those classified by respiration such as obligate aerobes, obligate anaerobes, facultative anaerobes, aerotolerant bacteria, and micoaerophiles. Other examples of bacteria include those classified by morphology such as coccus, bacillus, vibro, spirillum, spirochaete, and filamentous bacteria). Other bacteria include those classified by molecular datasuch as Actinobacteria (e.g., Actinomycetales, Bifidobacteriales), Firmicute (e.g., Bacilli, Clostridia, Mollicutes), Bacteroidete (Bacteriodetes, Flavobacteria), Chlamydiae (Chlamydiales), Fusobacteria, Proteobacteria (Alpha Proteobacteria, Beta Proteobacteria, Gamma Proteobacteria, Epsilon Proteobacteria), Spirochaete, or a combination thereof. In another embodiment, the archea is a cyanobacteria. In yet another aspect, the fungus is yeast. In still another aspect, the fungi is fusarium. In another aspect, the yeast is a Candida, such as Candida albicans.

Specific examples of bacteria include Actinomyces israelii, Actinomyces naeslundi, Actinomyces meyeri, Actinomyces odontolyticus, Actinomyces viscosus, Propionibacterium acnes, Tropheryma whipplei, Actinomadura madurae, Actinomadura pelletieri, Nocardiopsaceae, Nocardiopsis dassonvillei, Streptomyces somaliensis, Nocardia asteroids, Nocardia brasiliensis, Nocardia otitidiscaviarum, Nocardia transvalensis, Rhodococcus equi, Mycobacterium leprae, Mycobacterium tuberculosis complex, Mycobacterium tuberculosis, Mycobacterium africanum, Mycobacterium bovis, Mycobacterium avium complex (MAC), Mycobacterium avium, Mycobacterium intracellulare, Mycobacterium scrofulaceum, Mycobacterium fortuitum complex (MFC), Mycobacterium fortuitum, Mycobacterium chelonae, Mycobacterium kansasii, Mycobacterium ulcerans, Mycobacterium abscessus, Mycobacterium haemophilum, Mycobacterium marinum, Mycobacterium simiae, Mycobacterium xenopi, Corynebacterium diphtheria, Corynebacterium minutissimum, Corynebacterium jeikeium, Gardnerella vaginalis, Streptococcus pyogenes, Streptococcus agalactiae, Streptococcus dysgalactiae group, S. dysgalactiae, S. equi, Streptococcus equines, Streptococcus canis, Streptococcus pneumonia, Streptococcus viridans group (α-hemolytic or non-hemolytic), S. mitis, S. mutans, S. oralis, S. sanguinis, S. sobrinus, Streptococcus milleri group (Lancefield Group F), S. anginosus, S. constellatus, S. intermedius, Enterococcus faecalis, Enterococcus faecium, Staphylococcus aureus, Staphylococcus epidermidis, Staphylococcus saprophyticus, Bacillus cereus, Listeria monocytogenes, Clostridium botulinum, Clostridium difficile, Clostridium perfringens, Clostridium tetani, Mycoplasma genitalium, Mycoplasma pneumonia, Ureaplasma urealyticum, Erysipelothrix rhusiopathiae, Bacteroides fragilis, Tannerella forsythia, Porphyromonas gingivalis, Prevotella intermedia, Capnocytophaga canimorsus, Chlamydia trachomatis, Chlamydophila psittaci, Chlamydophila pneumonia, Fusobacterium necrophorum, Fusobacterium nucleatum, Fusobacterium nucleatum nucleatum, Fusobacterium nucleatum polymorphum, Streptobacillus moniliformis, Rickettsia, Rickettsia rickettsii, Rickettsia conorii, Rickettsia akari, Rickettsia-typhus group, Rickettsia typhi, Rickettsia prowazekii, Orientia tsutsugamushi, Anaplasma phagocytophilum, Ehrlichia chaffeensis, Brucella abortus, Bartonella bacilliformis, Bartonella henselae, Bartonella Quintana, Neisseria meningitides, Neisseria gonorrhoeae, Eikenella corrodens, Kingella kingae, Burkholderia pseudomallei group, B. pseudomallei, B. mallei, Burkholderia cepacia complex, B. cepacia, B. vietnamiensis, B. multivorans, B. stabilis, B. ambifaria, B. anthina, B. cenocepacia, B. dolosa, B. pyrrocinia, Bordetella pertussis, Bordetella parapertussis, Spirillum minus (Rat-bite fever), Enterobacter cloacae, Escherichia coli, Klebsiella granulomatis, Klebsiella oxytoca, Klebsiella pneumonia, Plesiomonas shigelloides, Proteus mirabilis, Proteus vulgaris, Salmonella enteric, Serratia marcescens, Shigella dysenteriae, Shigella flexneri, Shigella sonnei, Yersinia enterocolitica, Yersinia pestis, Yersinia pseudotuberculosis, Cardiobacterium hominis, Legionella pneumophila, Legionella longbeachae, Coxiella burnetii, Haemophilus influenza, Haemophilus ducreyi, Pasteurella multocida, Actinobacillus ureae, Actinobacillus hominis, Aggregatibacter actinomycetemcomitans, Pseudomonas aeruginosa, Moraxella catarrhalis, Acinetobacter baumannii, Francisella tularensis, Vibrio cholera, Vibrio vulnificus, Vibrio parahaemolyticus, Stenotrophomonas maltophilia, Campylobacter jejuni, Campylobacter coli, Campylobacter lari, Campylobacter fetus, Helicobacter pylori, Helicobacter cinaedi, Helicobacter fennelliae, Treponema pallidum, Treponema pallidum pallidum, Treponema pallidum endemicum, Treponema pallidum pertenue, Treponema carateum (pinta), Treponema denticola, Borrelia recurrentis, Borrelia burgdorferi, and Leptospira interrogans

Yet other examples of bacteria include acidogenic gram-positive cocci (e.g., Streptococcus) that is associated with dental caries, gram negative anaerobic oral bacteria that is associated with periodontis, nontypable strains of Haemophilus influenza that is associated with otitis media, gram positive cocci (e.g., staplyococcus) that is associated with musculoskeletal infections, Group A streptococci that is associated with necrotizing fasciitis, enteric bacteria (e.g., E. coli) that is associated with biliary tract infection, various and often mixed bacterial and fungal species that is associated with osteomyelitis, E. coli and other gram negative bacteria that is associated with bacterial prostatitis, viridians group streptococci that is associated with native valve endocarditis, P. aeruginosa and Burkholderia cepacia that is associated with cystic fibrosis pneumonia, pseudomonas pseudomallei is associated with meloidosis, gram negative rods associated with ICU pneumonia, Staphylococcus epidermis and S. aereus associated with nosocomial infections of sutures, S. epidermis and S. aureus associated with nosocomial infections of exit sites, S. epidermis and S. Aureua associated with nosocomial infections of ateriovenous shunts, gram positive cocci associated with nosocomial infections of schleral buckles, P. aeruginosa and gram-positive cocci associated with nosocomial infections of contact lens, E. coli and gram-negative rods associated with nosocomial infections of urinary catheter cystitis, bacteria and fungi associated with nosocomial infections of peritoneal dialysis peritonitis, actinomyces israelii associated with nosocomial infections of IUDs, bacteria and fungi associated with nosocomial infections of endotracheal tubes, S. epidermis and C. albicans associated with nosocomial infections of Hickman catheters, S. epidermis and others associated with nosocomial infections of central venous catheters, S. aureus and S. epidermis associated with nosocomial infections of mechanical heart valves, gram positive cocci associated with nosocomial infections of vascular grafts, enteric bacteria and fungi associated with nosocomial infections of bilary stent blockage, S. aureus and S. epidermis associated with nosocomial infections of orthopedic devices, and S. aureus and S. epidermis associated with nosocomial infections of penile prostheses.

In addition, to contacting the one or more microorganisms with mucin, one or more surfaces upon which the one or more microorganisms can attach to and/or form a biofilm on, can be contacted with purified, non-human mucin. As will be appreciated by those of skill in the art, the surface can be the surface of an individual (an internal or external surface such as a cavity or an orifice of an individual), a surface of a product that is ingested by an individual (e.g., food, nutraceutical, medicine, dental product, a hand cleaner), a surface of an instrument or device that comes in contact with an individual (e.g., for treatment, prosthetic and/or diagnostic purposes), a surface that comes in contact with water (e.g., all or portion of water treatment and/or purification system) or a combination thereof. Specific examples of such surfaces include an ear canal, an oral cavity (e.g., teeth for inhibition of dental plaque), a wound, a (one or more) suture, a prosthetic (e.g., limb, joint, pins, screws), a valve (e.g., a heart valve), live tissue, dead tissue (e.g., dead bone) of an individual; mouthwash, toothpaste, dental floss, contact lens; internal or external surfaces of a stent, shunt, catheter, endoscope, a swab (e.g., a Q-tip); floors, countertops; human work surfaces, such as doorknobs, table tops, faucet handles, toilets, phones, et cetera; internal and external surfaces of pipes used in drug, food and/or water treatment/processing/packaging chains and the like, as well as ductwork, and filters for ductwork, e.g., in environment control systems such as heating and air conditioning, e.g. enclosed spaces, such as in automobiles, trains, airplanes, subways, et cetera.

### EXEMPLIFICATION

### Example I

### Bacteria

### Experimental Procedures

### Strains and Growth Conditions

All strains, plasmids, and their sources are listed in the supplementary procedures. Pseudomonas aeruginosa PAO1 was the wild-type in this study. P. aeruginosa from the PA14 background was used for the motility mutants presented in FIG. Slh-j. E. coli wild-type strain was W3110, subtype ZK2686. The following media were used: lysogeny broth (LB), tryptone broth (TB; 10% w/v tryptone), Pseudomonas minimal medium (PMM; 2.5 mM Na-succinate, 1.2 mM MgSO4, 35 mM K2HPO4, 22 mM KH2PO4, 0.8 mM (NH4)2SO4, E. coli minimal medium (M63 salts) supplemented with 0.2% (w/v) glucose and 0.5% (w/v) casamino acids (M63+). Unless specified otherwise, the standard growth medium for P. aeruginosa was 1% mucin (w/v) in PMM. Mucins were dissolved in the medium by gentle shaking overnight at 4° C.

### Mucin Purification

An ideal source for purification of native MUC5AC is pig stomachs, which secrete MUC5AC that is homologous to the human glycoprotein [Turner, B. et al. (2007) FASEB J. 21]. Porcine gastric mucins were purified as described previously, with the omission of the CsCl density gradient centrifugation [Celli, J. et al. (2005) Biomacromolecules 6, 1329-33]. Mass spectrometry analysis was used to determine the composition of the mucin preparation as described previously [Lieleg, O. et al. (2012) www.ncbi.nlm.nih.gov/pubmed/22475261]. Briefly, the analysis was performed at the Harvard Microchemistry and Proteomics Analysis Facility by microcapillary reverse-phase HPLC nanoelectrospray tandem mass spectrometry on a Thermo LTQ-Orbitrap mass spectrometer. The spectra were analyzed using the algorithm Sequest [Yates, J. R. et al. (1995) Anal. Chem. 67, 1426-1436]. The analysis showed that MUC5AC was the predominant mucin present in our purified extract, which also contained MUC2, MUC5B and MUC6 as well as other proteins including histones, actin and albumin. In addition, its quality was tested by rheology as described in [Kocevar-Nared, J. et al. (1997) Biomaterials 18, 677-81; Celli, J. et al. (2005) Biomacromolecules 6, 1329-33], which confirmed that the isolated mucins displayed viscoelastic properties similar to native mucus.

### Microbial Adhesion Assays

For adhesion experiments, PAO1 wild-type and PAO1 ΔflgE were inoculated in LB and grown overnight at 37° C., shaking Overnight cultures were diluted 1:100 into PMM and grown shaking at 37° C. for 4 h. 1 ml of exponential phase cells (OD600=0.4 to 0.45) were centrifuged and cells were resuspended in 400 μL, sterile PMM. These cells were diluted 1:10 in PMM and then further diluted 1:10 into the medium to be tested (PMM only, 0.5% mucin, 0.5% PEG 3350, or 0.5% dextran). 40 μL, of this mixture was pipetted onto glass slides with shallow spherical depressions, covered with a glass coverslip and inverted. Pairs of images were taken 2 s apart in multiple fields for each sample at 10, 30, 50 and 70 min. Image pairs were compared to differentiate firmly attached cells from moving cells in each frame. Adherent cells were counted for each time point. Pairs of dividing cells were counted as single cells.

### Quantification of Biofilm Formation in Mucin Gels

Freshly growing cells at an OD600 of 0.01 were inoculated in polypropylene PCR tubes and incubated at 37° C. in TB or in TB containing 0.5% (w/v) mucins. After 6 h the planktonic cells were removed for quantification, and the adherent cells in the tubes were washed 2 times with PBS to remove non-adherent cells. Planktonic and adherent cells were stained with 5 mg/ml MTT (3-(4,5-Dimethylthiazol-2-yl)-2,5-diphenyltetrazolium bromide) for 2 h at 37° C., and subsequently destained with 20% sodium dodecyl sulfate in 50% dimethylformamide (adjusted to pH=4.7) overnight at 37° C. The resulting solutions were quantified using a plate reader (OD595).

### Particle Tracking

For measurement of cell velocities, bacteria were grown to exponential phase as described above. Cells were then stained with Syto9 live cell stain by adding Syto9 1:1000 into the culture and incubating for 10 minutes at room temperature. The stained cells were then diluted 1:10 into a 50% strength solution of growth medium (as indicated in FIG.) or growth medium supplemented with mucin, dextran or PEG. These solutions were mixed thoroughly and immediately dispensed into chambers for visualization. The chambers were constructed with glass microscope slides and coverslips treated with O₂ plasma for 5 min. Coverslips were adhered to slides using double-sided tape, forming chambers approximately 8 mm wide, 18 mm long and 60 μm tall. Once the chambers were filled, they were sealed with silicone vacuum grease to prevent evaporation. Videos of cells were taken on an inverted fluorescent microscope at 20 frames per second. Four videos of 20 s each were acquired within 5 min. of loading of the chamber. Videos were analyzed in ImageJ using the Mosaic particle tracking plugin [Sbalzarini, I. F., and Koumoutsakos, P. (2005) J. Struct. Biol. 151, 182-195]. The trajectories obtained were then processed using Matlab to determine velocities and diffusivities. Diffusivities were based upon mean squared displacement values for a range of lag times. Trajectories were also examined visually to ensure accuracy.

### Antibiotic Treatment

To determine the antibiotic resistance of flocs grown in mucin-media, cells were grown in PMM with 1% (w/v) mucin. After 20 h, the number of cells was determined by counting CFU; this number was used as the reference number prior to treatment. The antibiotics ofloxacin and colistin were added to the cultures at final concentrations of 20 μg/ml, and the cultures were grown at 37° C. for 3 h. After treatment, the number of survivors was estimated by measuring the CFU. To avoid aggregates, each sample was bead-bashed for 30 s before diluting and plating. Each experiment was carried out in triplicate. To determine the resistance of cells grown in the absence of mucins, an exponential phase culture was adjusted to contain the same number of cells as had grown in 1% mucin in 20 h, and challenged with antibiotics as described above.

### Floc Size Measurements

To observe cells growing in mucins, cells from exponential growth phase were added to a 1% mucin gel so that the final concentration was 50-100 cells μl-1. The mix was placed in a 96-well glass bottom plate (MatTek) and incubated at 37° C. for 20 h. Images were taken immediately after incubation using an Axiovert 200M (Zeiss). To quantify the dimensions of each colony in a given picture, we used the software ImageJ. We first subtracted the background using the rolling ball algorithm. The image was then thresholded using an iterative procedure based on the isodata algorithm. A group of cells was defined as a floc when it was composed of at least 2-3 cells. Analysis with Minitab 16 (Minitab Inc.) showed that the data were not normally distributed. The data was plotted using box-plots to provide an unbiased overview of the distribution of the floc sizes for each condition. Four pictures from three independent experiments were analyzed for each strain.

### Constructions of Deletion Mutants and GFP-Labeled Strains

Deletions in P. aeruginosa strains were obtained using Splicing Over Extension (SOE)-PCR, as described previously [Schweizer, H. P. Mol Microbiol 6, 1195-204 (1992)], and confirmed by PCR. In addition, the inability of the strains ΔflgE, ΔflgK, ΔfliD to swim and swarm, and the incapacity of ΔpilB to twitch, was tested as described previously [O'Toole, G. A. & Kolter, R. Molecular Microbiology 30, 295-304 (1998)]. To facilitate microscopy, GFP was expressed constitutively in each strain. GFP expressing strains were created as described previously [Lambertsen, L. et al. Environ Microbiol 6, 726-32 (2004)]. Strains from the PA14 background used in FIG. Slh-j that express GFP were grown in the presence of carbenicillin (250 μg/ml) as described in Amiel, E. et al. Infect Immun 78, 2937-45 (2010). E. coli ΔfliC was generated using P1 phage transduction [Thomason, L. C. et al. Curr Protoc Mol Biol Chapter 1, Unit 1.17 (2007)] from strain JW1908-1 obtained from the Keio collection (Coli Genetic Stock Center) into the ZK2686 background. Deletion of fliC was confirmed by PCR and motility agar assay.

### Generation of Complementation Plasmids

A complementation plasmid containing the flgE gene was created using the plasmid pMQ80 [Shanks, R. M. Q. et al. Appl Environ Microbiol 72, 5027-5036 (2006)]. Briefly, the GFP gene was removed from the plasmid via restriction enzyme digestion with ecoRI and hindIII. The flgE gene was amplified from P. aeruginosa PAO1 genomic DNA with primers containing ecoRI and hindIII restriction sites upstream and downstream of flgE respectively. After digestion, the flgE gene was inserted into the plasmid using T4 DNA ligase. For control plasmids, pMQ80 was treated with Klenow polymerase and blunt end-ligated without an insert. The plasmids were transformed [Choi, K.-H. et al. J. Microbiol. Methods 64, 391-397 (2006)] into E. coli DH5α cells and plated on LB agar containing 30 μg/mL gentamicin selective media. Resulting colonies were inoculated into overnight cultures and the plasmids were extracted using the GenElute™ Plasmid Miniprep Kit (Sigma Aldrich). The plasmids were transformed into PAO1 strains plated on LB agar plates containing 50 μg/mL gentamicin. Successful transformants were again transformed with the plasmid pSMC21 [Amiel, E. et al. Infect Immun 78, 2937-45 (2010)], a constitutive GFP expressing plasmid, to facilitate observation of floc formation (note: the fluorescent strains used in the rest of the study are gentamicin resistant and could not be used with pMQ80 which relies on gentamicin selection). Transformants were selected on LB agar containing 400 μg/mL carbenicillin and 50 μg/mL gentamicin. Floc formation in mucin was performed as described in the main text, with the addition of antibiotics (carbenicillin for GFP expression and gentamicin for complementation plasmids) and 100 mM arabinose to induce pMQ80 complementation vector expression.

List of Strains and Plasmids Used in this Study

| | | |
|---|---|---|
| Strains and plasmids | Description | Reference or Source |

E. coli

| | | |
|---|---|---|
| DH10B | F-endA1 recA1 galE15 galK16 nupG rpsL | Invitrogen |
| | ΔlacX74 Φ80lacZΔM15 araD139 | |
| | Δ(ara, leu)7697 | |
| | mcrA Δ(mrr-hsdRMS-mcrBC) λ- | |
| SM10 λpir | Kmᵣ, thi-1, thr, leu, tonA, lacY, supE, | Lambertsen, L. et |
| | recA::RP4- | al. Environ |
| | 2-Tc::Mu, pir+ pUX-BF13 (Apᵣ-Tn7 helper) | Microbiol 6, |
| | | 726-32 (2004) |
| S17-1 λpir | Tpᵣ Smᵣ recA, thi, pro, hsdR-M₊RP4: 2- | Mark Silby |
| | Tc:Mu: | |
| | Km Tn7 λpir | |
| S17-1 λpir + gfp | Tpᵣ Smᵣ recA, thi, pro, hsdR-M₊RP4: 2- | Wook Kim |
| | Tc:Mu: | |
| | Km Tn7 λpir + pBKminiTn7-Gm/Cm-gfp | |
| ZK2686 | W3110, Δ(argF-lac) U169 | Danese, P. N. et |
| | | al. Mol. |
| | | Microbiol. 37, |
| | | 424-432 (2000) |
| JW1908-1 | BW25113, F-, Δ(araD-araB)567, | CGSC |
| | ΔlacZ4787 (::rrnB-3), λ-, ΔfliC769::kan, rph- | |
| | 1, | |
| | Δ(rhaD-rhaB)568, hsdR514 | |
| ΔfliC | ZK2686, ΔfliC769::kan | This study |

P. aeruginosa

| | | |
|---|---|---|
| PAO1 | wild type, clinical isolate | Holloway, B. W. J Gen |
| | | Microbiol 13, 572-81 |
| | | (1955) |
| ΔflgE | PAO1-ΔflgE | This study |
| ΔfliD | PAO1-ΔfliD | This study |
| ΔflgK | PAO1-ΔflgK | This study |
| ΔpilB | PAO1-ΔpilB | This study |
| ΔflgE ΔpilB | PAO1-ΔflgE ΔpilB | This study |
| ΔalgD | PAO1-ΔalgD | Whitchurch, C. B. et al. J |
| | | Bacteriol 184, 4544-4554 |
| | | (2002) |
| WFPA800 | PAO1-Δpsl | Whitchurch, C. B. et al. J |
| | | Bacteriol 184, 4544-4554 |
| | | (2002) |
| ΔalgD ΔflgE | PAO1-ΔalgD ΔflgE | This study |
| Δpsl ΔflgE | PAO1-Δpsl ΔflgE | This study |
| PA14 | wild type, clinical isolate + | Amiel, E. et al. Infect |
| | pSMC21 | Immun 78, 2937-45 (2010) |
| PA14-ΔflgK | PA14-ΔflgK + pSMC21 | Amiel, E. et al. Infect |
| | | Immun 78, 2937-45 (2010) |
| PA14-ΔmotAB ΔmotCD | PA14-ΔmotAB ΔmotCD + | Amiel, E. et al. Infect |
| | pSMC21 | Immun 78, 2937-45 (2010) |

Plasmids

| | | |
|---|---|---|
| pMQ30 | pEX18Gm + CENURA, Gmᵣ, | Hoang, T. T. et al. Gene |
| | allelic replacement | 212, 77-86 (1998) |
| | vector | |
| pBKminiTn7- | Gmᵣ, Cmᵣ, transposon delivery | Lambertsen, L. et al. |
| Gm/Cm-gfp | plasmid | Environ Microbiol 6, 726- |
| | | 32 (2004) |
| pSMC21 | Apᵣ, Kanᵣ, Carbᵣ, plasmid | Amiel, E. et al. Infect |
| | containing GFP under | Immun 78, 2937-45 (2010) |
| | the control of P_{tac} constitutive | |
| | promoter | |
| pMQ80 | Complementation plasmid, GmR | Shanks, R. M. Q. et al. |
| | | Appl Environ Microbiol |
| | | 72, 5027-5036 (2006) |
| pMQflgE | Complementation plasmid | This study |
| | containing flgE | |

CGSC, Coli Genetic Stock Center (Yale)

### Motility Assay

M63 motility plates supplemented with 0.2% glucose, 1 mM MgSO4 and 0.5% casamino acids were created as described previously [O'Toole, G. A. & Kolter, R. Mol Microbiol 28, 449-61 (1998)]. To induce expression from the complementation plasmid, 100 mM arabinose was added to the plates. Wooden inoculation sticks were dipped into overnight cultures of the strains being tested and used to stab the center of the motility plates. The plates were incubated overnight (16 h) at 30° C.

### Statistical Analysis

Values are reported in the text as value±s.e.m. For statistical comparisons between groups with approximately normal distributions, the Student's two tailed t-test was used. Error bars in FIGS. are either standard deviation or s.e.m., as indicated in the legends.

### Results and Discussion

Mucin biopolymers prevented bacterial adhesion to underlying substrates. Bacterial motility was maintained or increased in the presence of mucins. Mucins blocked aggregate formation by motile bacteria. Immotile Pseudomonas aeruginosa formed alginate and Psl-dependent flocs in mucus

Mucins Reduce Surface Adhesion and Biofilm Formation of P. aeruginosa

To begin to dissect mucin-bacterial interactions, an in vitro assay that uses defined concentrations of native mucins was developed. As a source of mucins, native porcine gastric mucus was purified to obtain an extract composed predominantly of MUC5AC, which is one of the major gel-forming components in the lungs and stomach [Schade, C. et al. (1994) Gastroenterology 107, 180-188]. Natively purified mucins were used for this assay, as commercially available mucins are processed and have lost the ability to form viscoelastic hydrogels, as are generated by the native polymers [Waigh, T. A. et al. (2002) Langmuir 18, 7188-7195; Kocevar-Nared, J. et al. (1997) Biomaterials 18, 677-81; Crater, J. S., and Carrier, R. L. (2010) Macromolecular Bioscience 10, 1473-1483]. The mucins were presented in solution, as they exist in the secreted lung mucus, instead of depositing them onto a surface. Surface deposition of mucins is likely to adsorb functional groups, thereby partially dehydrating and altering the biochemical activity of the polymer.

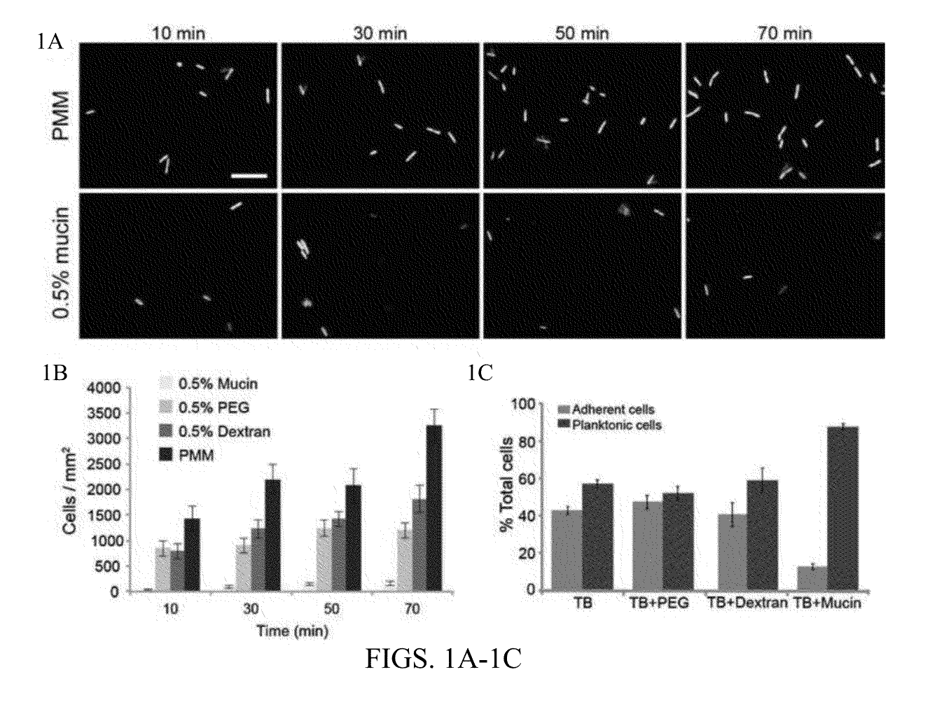

First tested was the effect of mucins on the ability of bacteria to colonize an immersed surface. A glass coverslip was suspended in culture medium that contained physiological concentrations of mucins [Kirkham, S. et al. (2002) Biochem. J. 361, 537-546]. Using the motile, opportunistic pathogen Pseudomonas aeruginosa, firm attachment was quantified by placing exponential-phase cells in contact with the coverslip and imaging using phase contrast microscopy. Cells that adhered to the surface and fully arrested (based on overlaying pairs of images separated by 2 s) were considered firmly attached, and were counted at 20-minute intervals (FIG. 1A).

It was found that mucins reduced bacterial surface adhesion by 20-fold over a 70 minute period (FIG. 1B). To test if this inhibitory effect was specific to the mucins, or a generic result of the presence of polymers, the effects of mucins was compared to solutions of polyethylene glycol (PEG), a polymer often used as an anti-adhesive coating [Banerjee, I. et al. (2011) Advanced Materials 23, 690-718], and dextran, a branched, high molecular weight polysaccharide. In comparison to mucins, PEG and dextran demonstrated only mild reductions in bacterial adhesion at these early time points, indicating that mucins have singular effects that cannot be attributed to their polysaccharide or soluble polymeric attributes alone. At 6 h, a time at which biofilms have begun to form, approximately 90% of P. aeruginosa cells remained planktonic in the presence of mucins, compared with 50-60% in tryptone broth (TB) alone or TB plus PEG or dextran (FIG. 1C).

### Mucin Gels Maintain or Augment Bacterial Swimming Motility

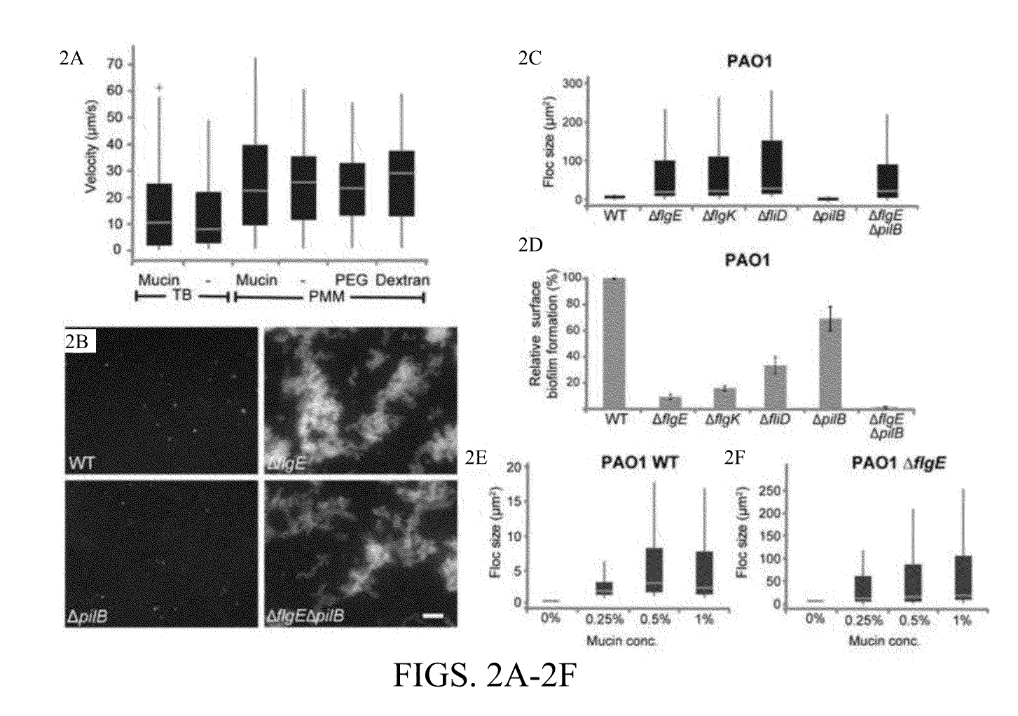

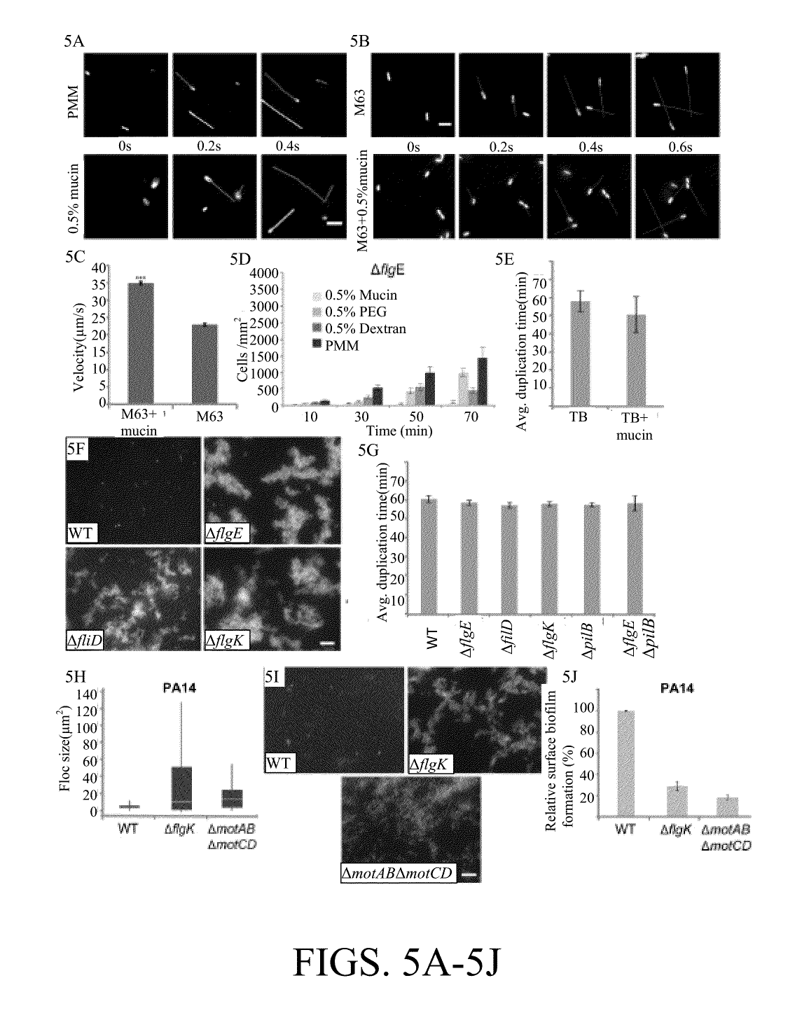

It is tempting to speculate that bacteria failed to access the underlying surface because they were trapped within the mucin network [Matsui, H. et al. (2006) PNAS 103, 18131-18136]. If this is true, a measurable decrease of motility within the mucin hydrogel should be seen. First, to test if motion was hindered in the presence of mucins the movements of P. aeruginosa cells that carried a deletion in the flagellar hook gene (flgE), and were thus deficient in self-propulsion, were tracked. These cells demonstrated a significant decrease in diffusivity (p<0.001) in mucin environments, from 2.4±0.2×10⁻⁹ cm²/s to 1.0±0.1×10⁻⁹ cm²/s (n≧96 cells), reflecting a higher apparent viscosity of mucin-containing gels, and suggesting that geometric hindrance was present. However, the wildtype cells remained highly motile in the presence of the mucins. The distribution of velocities of swimming cells in mucins was similar to that in liquid medium, despite the differences in apparent viscosity (FIG. 2A, 5A). This effect was apparent when cells in Pseudomonas minimal medium (PMM) as well as in tryptone broth (TB) with or without mucins were compared. To test whether this effect was specific for Pseudomonas, or if it was a more general phenomenon that affects other swimming bacteria, a different motile bacterium, Escherichia coli, was tracked. Despite a significant decrease in diffusivity (p<0.001) of deflagellated cells (ΔfliC) in mucins, from 2.2±0.2×10⁻⁹ cm²/s to 0.7±0.1×10⁻⁹ cm²/s (n≧92 cells), the wildtype cells had significantly increased swimming velocities in mucins compared with mediumonly (FIGS. 5B, 5C).

Immotile P. aeurginosa Cells can Form Suspended Flocs in Mucin Gels

If mucins can prevent surface colonization by maintaining cellular motility, it was speculated that cells lacking motility could overcome this dispersion effect and succeed in adhesion and biofilm formation in mucin environments. This line of inquiry has direct physiological relevance, as isolates of P. aeruginosa from cystic fibrosis (CF) mucus are often non-motile [Luzar, M. A. et al. (1985) Infect Immun 50, 577-82; Mahenthiralingam, E. et al. (1994) Infect. Immun. 62, 596-605], and the benefits of this are not well understood. As with the wild-type, mucins detectably reduced surface adhesion of non-motile cells (ΔflgE), which are already poorly adherent (FIG. 5D; compare to FIG. 1B). To look beyond surface adhesion in the presence of mucin, the bacteria in the volume of the mucin gel after 20 h of incubation was observed. The wild-type cells remained largely as individual cells or small, suspended colonies (FIGS. 2B, 2C) of up to 20 μm² (this corresponded roughly to clusters of 10-20 cells) distributed throughout the volume of the mucin medium. Increasing mucin concentration did not visibly increase cellular cluster size (FIG. 2E). However, when observing the ΔflgE mutant, a striking difference was noticed compared to the behavior of wild-type cells. The flagella mutant formed large aggregated flocs of up to 250 μm² (FIG. 2B, 2C). These differences were not likely due to variations in cellular densities in the mucin medium, as the strain ΔflgE displayed similar growth rates in the presence of mucins (FIG. 5E). A similar behavior was found for two additional flagella mutants, ΔflgK, which lack a hook filament junction protein, and ΔfliD, which lack an adhesive protein at the tip of the flagellar filament (FIGS. 2B, 2C and 5F) but not for ΔpilB which lack pilus-mediated adhesion and twitching motility (FIGS. 2B, 2C). The ability of cells to form suspended flocs was inversely correlated with their ability to form surface biofilms in mucin-free environments (FIG. 2D). For example, wildtype and ΔpilB cells formed substantial surface biofilms in the absence of mucins, but failed to form large suspended flocs in the presence of mucins. Conversely, the various flagellar mutants formed large flocs, but had reduced surface biofilms in the absence of mucins. All mutants tested displayed similar growth rates (FIG. 5G). The flocs formed by ΔflgE strains increased in maximum size with increasing mucin concentration (FIG. 2F).

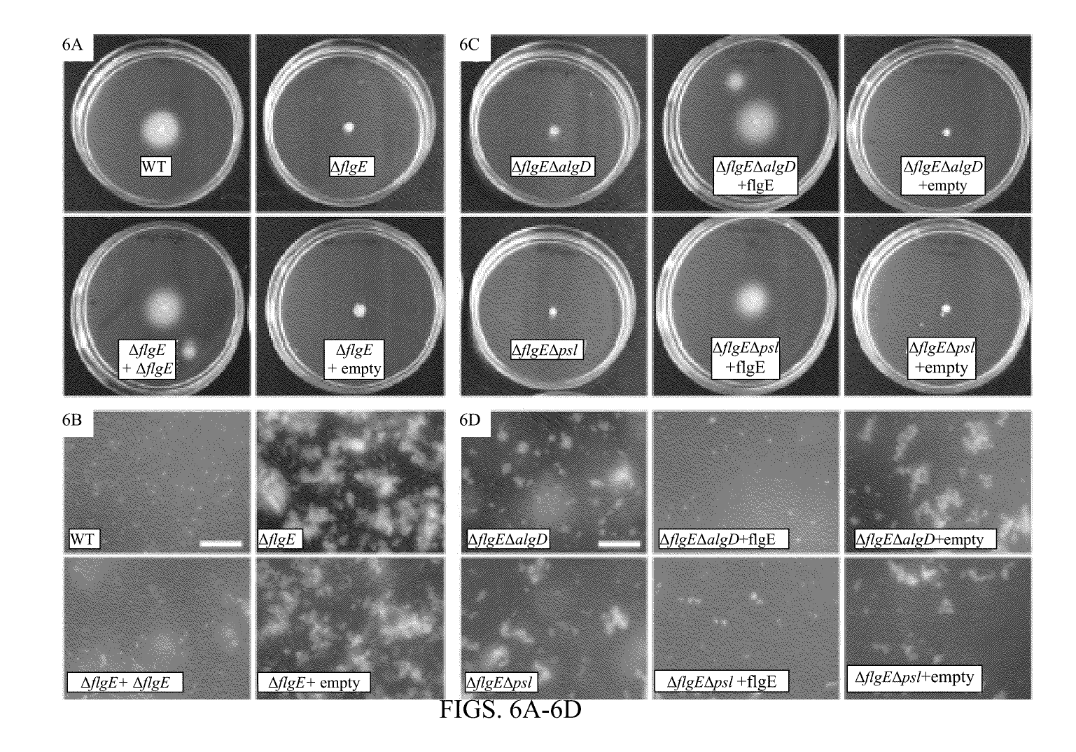

It was hypothesized that loss of flagellar motility (rather than other properties of flagella, such as adhesion) was the dominant contributor to the observed aggregation. To test this, mucin-dependent flocculation was measured by a PA14 strain that carried a fully assembled flagellum, but was paralyzed due to deletions in all four stators in the motor complex (ΔmotABΔmotCD). This mutant formed substantially larger flocs (up to 60 μm²) than the wild-type (FIGS. 5H, 5I), but the structures were smaller than those formed by the ΔflgK strain. Again, floc-forming ability in mucins tended to be negatively correlated with surface biofilm formation in medium-only environments (FIG. 5J). Both a loss of motility and loss of the flagella itself, therefore, appeared to contribute to mucin colonization. Complementing the flgE deletion in PAO1 ΔflgE restored swimming motility and diminished the capacity of the bacteria to form flocs in mucin, indicating that it is indeed the lack of flagella that caused the formation of flocs (FIGS. 6A-6D).

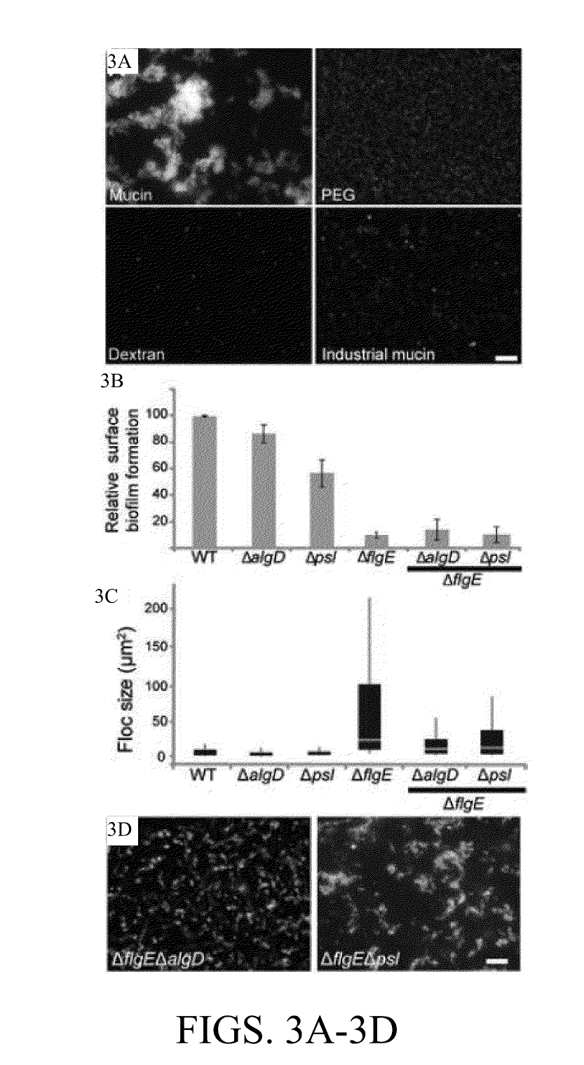

The data herein indicate that mucins are highly effective at preventing swimming cells from surface attachment and from forming suspended aggregates. Previous work has indicated that fliD is an adhesin for mucin [Arora, S. K. et al. (1998) Infect Immun 66, 1000-7], yet it does not appear to be required for the aggregative phenotype (FIG. 2C). How then do the flagella mutants achieve aggregate formation? It appeared that their lack of motility enables cells to form clonal outgrowths of individual cells within the mucin. This was supported by culturing mixtures of fluorescent and non-fluorescent immotile cells in mucin medium. Over the course of 20 h small homogeneous patches of ten to twenty cells emerged and further expanded (FIGS. 6A-6D). Notably, floc formation did not occur in PEG, dextran or industrially purified mucins (FIG. 3A). It appeared that this phenomenon depended on specific features unique to native mucins.

P. aeruginosa Floc Formation is Dependent on the Production of Psl- and Alginate

Flagella loss appears to allow bacteria to effectively colonize mucus in a manner reminiscent of surface attached biofilms. Just how similar are these two forms of bacterial aggregation? To address this, whether floc formation by non-motile cells required extracellular matrix, a hallmark of biofilms, was tested. Specifically, Psi, which plays a structural role in the maturation of surface-attached biofilms [Ma, L. et al. (2006) J Bacteriol 188, 8213-21] and alginate, which appears to play only a minor role in biofilm formation (FIG. 3B, [Wozniak, D. J. et al. (2003) Proc Natl Acad Sci USA 100, 7907-12]) but is overexpressed in colonies adapted to growth in CF lung mucus, were investigated [Hentzer, M. et al. (2001) J Bacteriol 183, 5395-401; Stapper, A. P. et al. (2004) J Med Microbiol 53, 679-90; Hoffmann, N. et al. (2007) Antimicrob Agents Chemother 51, 3677-8]. Using previously characterized single algD and psl mutant strains [Ma, L. et al. (2006) J Bacteriol 188, 8213-21; Whitchurch, C. B. et al. (2002) J Bacteriol 184, 4544-4554], additional flgE mutations were introduced to study the importance of the extracellular matrix on the immotile flocs. Complementation of the double mutants with flgE was able to restore motility (FIGS. 6A-6D). It was found that both polymers, particularly alginate, were important for floc formation (FIGS. 3C, 3D). This phenotype may be relevant to CF pathology, where the formation of P. aeruginosa flocs inside the lung mucus is associated with the rise of antibiotic resistance [Moreau-Marquis, S. et al. (2008) Pul Pharm 21, 595-599]. In sum, the data herein indicate that mucin-based flocs and biofilms have the same broad reliance on extracellular matrix but the mechanistic details differ in important ways. Specifically, flocs rely on alginate and flagella loss in a manner not seen in surface attached biofilms.

P. aeruginosa Flocs that Emerge in Mucin Gels are Antibiotic Resistant

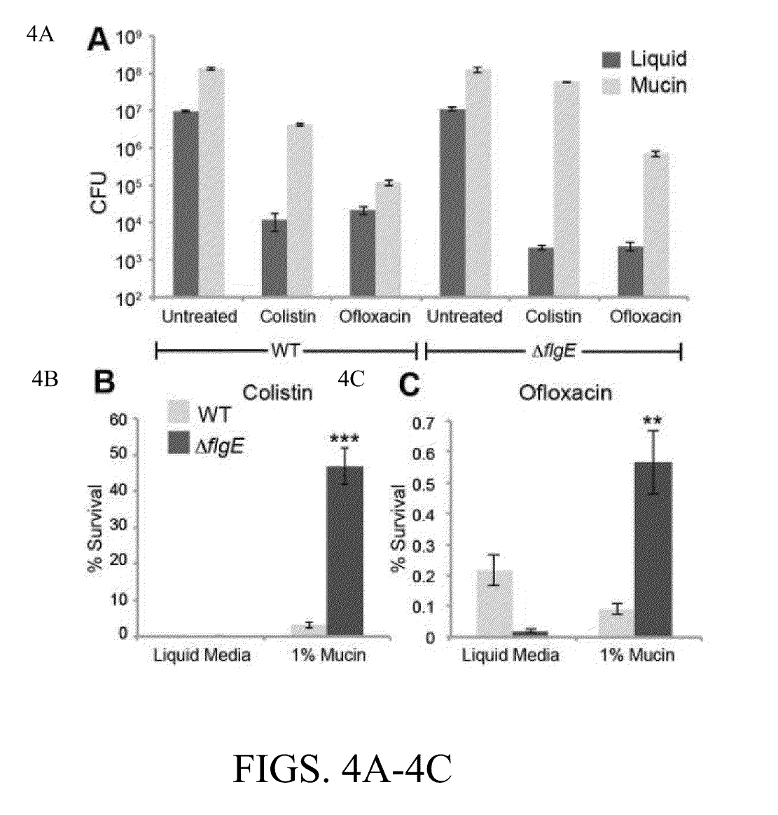

Whether floc formation can provide bacteria with a selective advantage was also investigated. Again by analogy with biofilms, it was hypothesized that the immotile cellular aggregates that emerge in the presence of mucins also have a higher resistance toward antibiotics. Wild-type and the non-motile ΔflgE cells were grown in mucin media for 20 h, and then both strains were subjected to two clinically relevant antibiotics that differ in their mode of action (FIGS. 4A-4C). This experiment revealed two points: first, both wild-type and ΔflgE bacteria were systematically more resistant to colistin in the presence of mucins as compared to liquid culture without mucins. This indicates that the mucins themselves have the capacity to reduce the efficacy of colistin, regardless of whether cells are planktonic (wild-type) or form flocs (ΔflgE). Second, it appeared that the floc-forming ΔflgE cells were more resistant to both antibiotics in the mucin medium than the motile wild type cells. The percent survival of the bacteria in either condition was determined, by normalizing to the cell numbers in the untreated samples in liquid and mucin. Inside the mucin medium, the non-motile flagella mutants were on average 14 times more resistant to colistin (FIG. 4B) and approximately 6 times more resistant to ofloxacin (FIG. 4C) than wild-type cells, both of which are statistically significant differences. It was concluded that the aggregates that emerge upon loss of motility indeed have an increased resistance compared to motile wild-type cells, likely due to the presence of an altered composition or quantity of extracellular matrix components, or due to a protective effect of increased cell density [Connell, J. L. et al. (2010) mbio.asm.org/content/1/4/e00202-10 [Accessed Sep. 16, 2012]].

### Conclusions and Outlook

Shown herein is that animals provide a candidate solution to inhibit biofilm formation, namely mucin polymers. Critically, the results provided herein demonstrate that mucins can limit bacterial surface attachment and biofilm formation without killing or trapping bacteria, which helps to limit selective pressure for resistance. Indeed, the only evidence for a resistance phenotype comes in the form of non-motile cells, which are likely to be strongly limited in other modes of virulence [Luzar, M. A. et al. (1985) Infect Immun 50, 577-82; Josenhans, C., and Suerbaum, S. (2002) Int. J. Med. Microbiol. 291, 605-614]. The observations of motility and reduced adhesion in mucin media provided herein are similar to findings for Campylobacter jejuni in mouse intestinal crypts. In a previous study, extracted epithelial scrapings from C. jejuni-colonized gnotobiotic mice demonstrated a lack of adhesion and unhindered motility within the crypts [Lee, A. et al. (1986) Infect. Immun. 51, 536-546]. Similar to this, a recent study showed that when supplemented in agar plates, mucins appear to increase motility of P. aeruginosa [Yeung, A. T. Y. et al. (2012) Available at: mbio.asm.org/content/3/3/e00073-12 [Accessed May 11, 2012]]. At first sight these and the findings herein contrast with reports on surface-immobilized mucins, which arrest [Arora, S. K. et al. (1998) Infect Immun 66, 1000-7; Vishwanath, S., and Ramphal, R. (1984) Infect Immun 45, 197-202] and can cause large aggregate formation of P. aeruginosa cells [Landry, R. M. et al. (2006) Molecular Microbiology 59, 142-151]. However, these findings can be reconciled if one considers that the effects of mucins on motility may depend on their native three dimensional structure and hence biophysical properties such as viscoelasticity and lubricity, which are preserved in native mucus and presumably inside agar gels, but not when adsorbed to a two-dimensional surface [Yeung, A. T. Y. et al. (2012) Available at: mbio.asm.org/content/3/3/e00073-12 [Accessed May 11, 2012]]. The gel-forming mucin MUC2 has an ordered repeating ring structure [Ambort, D. et al. (2012) Proc. Natl. Acad. Sci. U.S.A. 109, 5645-5650], and it is speculated herein that also other gel-forming mucins, such as the MUC5AC used in the experiments described herein, display three dimensional features that affect their interactions with bacteria. Indeed, Berg and Turner have observed that certain structured viscous solutions allow increased velocities of motile bacteria by providing a rigid framework for generating propulsive forces [Berg, H. C., and Turner, L. (1979) Nature 278, 349-351].

### Example II

### Fungi

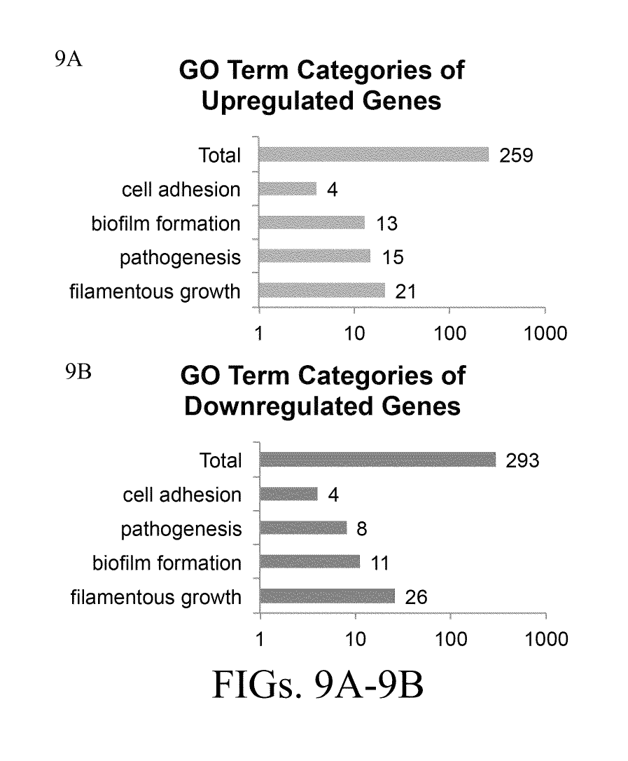

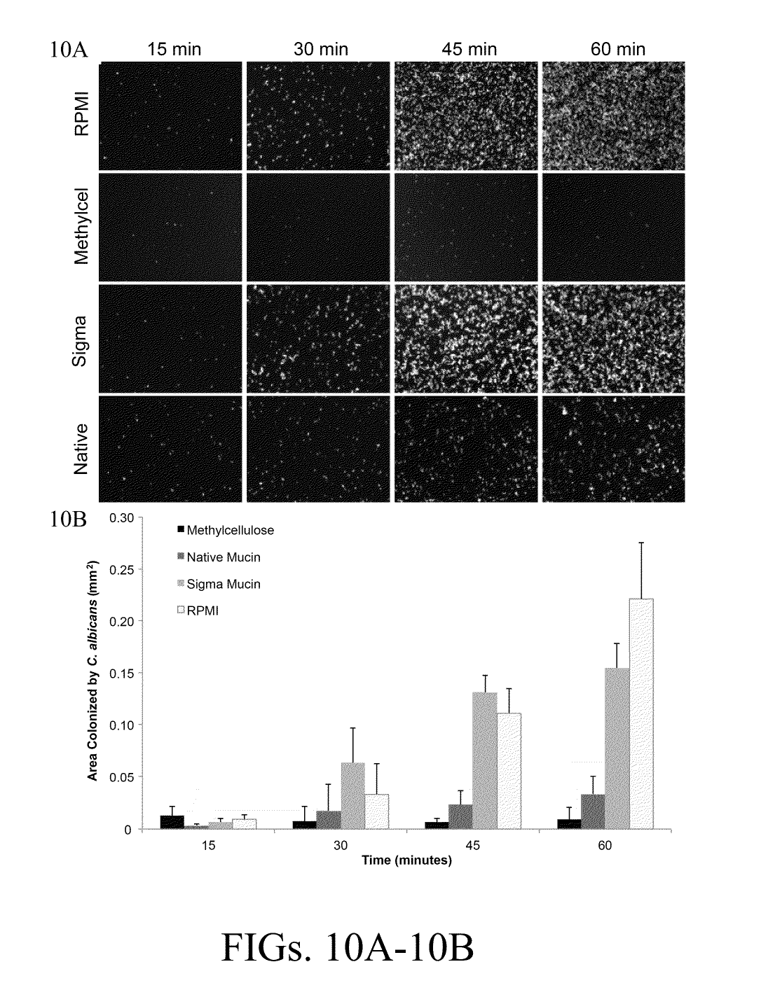

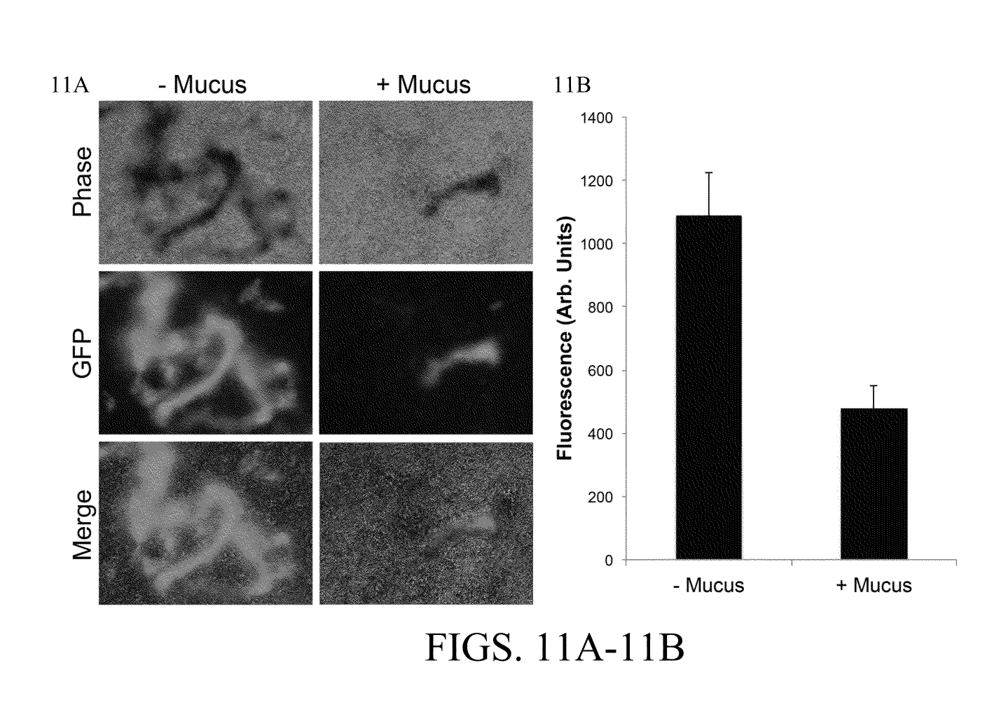

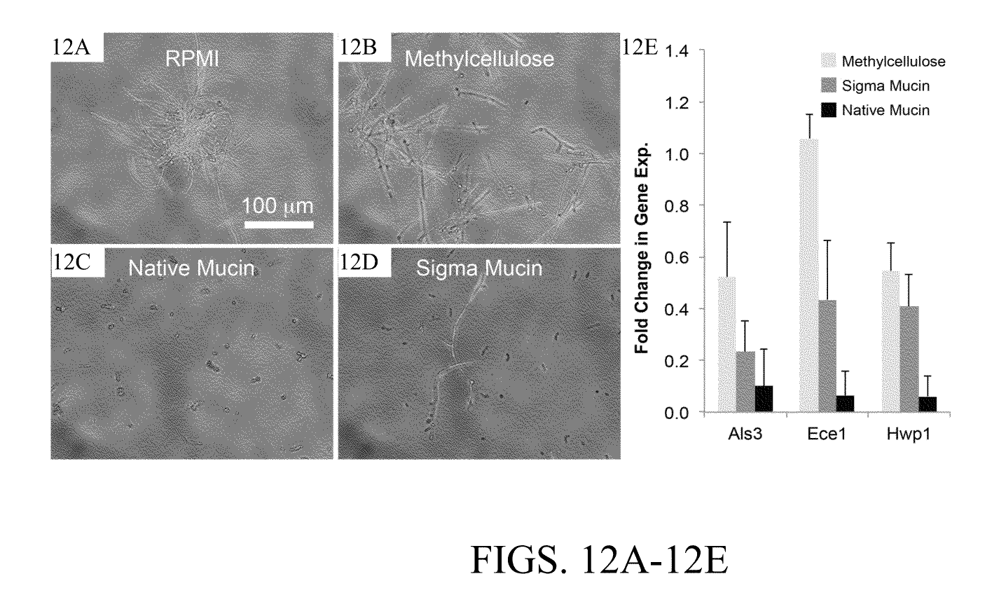

Here we show that mucins are able to suppress adhesion of the yeast Candida albicans to surfaces, as we previously showed with bacteria. The antimicrobial affects of mucins go even further than suppressing adhesion by modulating the expression of virulence genes. This includes the suppression of filamentation, a morphological form implicated in the invasion of epithelial cells during the infection process. Results are summarized in FIGS. 9-12.

### Experimental Procedures

C. albicans Strains and Media:

All strains were maintained on YPD agar (2% Bacto peptone, 2% dextrose, 1% yeast extract, 1.5% agar) and grown at 30° C. Single colonies were inoculated into YPD and grown with shaking overnight before experiments were performed.

The experiments were performed using YPD or RPMI 1640 (Gibco 31800-089) buffered with 165 mM MOPS and supplemented with 0.2% NaHCO3 and 2% glucose. 0.5% methylcellulose was prepared from a 5% stock solution by dilution in RPMI. Type II mucin from porcine stomach (Sigma), was dialyzed in MilliQ H₂O in a Spectra/Por Float-A-Lyzer G2 dialysis tube with a 100 kDa molecular weight cutoff; this PGM was dissolved in RPMI and vortexed at 4° C. overnight. 0.5% native PGM was prepared by dissolving PGM in RPMI and vortexing at 4° C. overnight.

The C. albicans yeast strains used were SC5314, MLR62 and HGFP3. Strain MLR62 (3) was constructed by linking GFP to the constitutive TEFL promoter and was provided by A. Mitchell (Carnegie Mellon University, Pittsburgh, Pa., USA) Strain HGFP3 (4) was constructed by inserting the GFP gene next to the promoter of HWP1, a gene for a hyphal cell wall protein, and was provided by E. Mylonakis (Massachusetts General Hospital, Boston, Mass., USA) with permission of P. Sundstrom (Stabb et al., 2003).

Extraction of RNA and Creation of cDNA

1 mL of RPMI or PGM in RPMI was inoculated with 10 μL of an overnight culture of strain SC5314 in a culture tube and incubated at 37° C. and 180 rpm for 8 h. RNA was extracted using the Epicentre® MasterPure™ Yeast RNA Purification Kit and treated with Sigma-Aldrich® AMPD1 Amplification Grade DNase I. 500 ng of RNA per sample was then reverse-transcribed to cDNA using the Invitrogen™ Superscript® III First-Strand Synthesis System for RT-PCR. cDNA samples were stored at −80° C. until use.

### Microarray

The gene expression microarrays were custom-designed oligonucleotide microarrays, containing at least two independent probes for each ORF from the C. albicans Assembly 21 genome and printed by Agilent Technologies (AMADID #020166). The microarrays were hybridized by C. Nobile from the Johnson Lab at the University of California, San Francisco.

The arrays were analyzed by the BioMicro Center of MIT. The microarray data was processed using the Limma package in R. Fluorescence signals from the arrays for the different conditions were visualized for the green and red channels of all the arrays. The data were first background-corrected for each array using the “normexp” algorithm with an offset of 50 (to “dampen” spots with very low expression values), followed by a loess-based within-array correction. The arrays were then quantile-normalized within each condition. Gene expression was modeled using a linear model and a design matrix accounting for the labeling scheme, and an empirical Bayesian statistics for gene expression was calculated for each gene in RPMI and FBS+YPD conditions. P-values were adjusted using the Benjamini-Hochberg (BH) procedure; BH-adjusted p-values<0.1 were considered significant. An additional filter on absolute fold-changes of at least 1.5 was then applied to the FBS+YPD samples.

Gene ontology analysis was performed on genes that were up- or down-regulated in both conditions separately, and then on the intersections of the RPMI and FBS+YPD differentially-regulated gene sets, using the Candida genome database.

### Quantitative PCR

Primers were designed for each gene and obtained from Sigma®. 2 μL of cDNA, 5 μM forward primer, 5 μM reverse primer, 6 μL dH₂O, and 10 μL Bio-Rad iQ™ SYBR® Green Supermix were mixed in wells of a Roche LightCycler® 480 White 96-well Multiwell Plate. Plates were centrifuged briefly to remove air bubbles. Quantitative PCR was carried out using a Roche LightCycler® 480 II machine with the following run protocol: (1) 95° C. for 3 min, (2) 40 cycles of 95° C. for 10 sec, 58° C. for 30 sec, and 72° C. for 30 sec, and (3) then slowly increased to 95° C. for melting curve analysis. Crossing threshold (CT) values were obtained and used for analysis.

### Polystyrene Attachment Assay

Polystyrene 96 well plates were inoculated with 100 μL of RPMI, 0.5% methylcellulose, 0.5% Sigma-manufactured PGM, and 0.5% native PGM containing 1 μL of an overnight culture of C. albicans SC5314. The plates were incubated statically at 37° C. Every 15 minutes, a time point was taken by washing the wells with 200 μL of PBS twice and then adding 100 μL of PBS. After 1 hour, 1 μL of 1 mg/mL Calcofluor white solution was added to each well. The samples were imaged with a Zeiss Observer Z1 inverted fluorescence microscope with a Zeiss Plan-Apochromat 10× objective. The experiment was performed in triplicate with 5 pictures taken of each well. The images were analyzed in ImageJ (4) as follows: Each image was converted to 8-bit and the contrast was enhanced (0.4% saturated pixels). The image was then thresholded to make a binary image. The image was then analyzed using the “Analyze Particles” tool to measure the surface area covered by cells. The surface areas of the 15 images for each condition and timepoint were averaged.

### Attachment to Human Mucus-Secreting Lung Cells (HT29-MTX)

HT29-MTX Mucus-secreting cells were grown as previously described (2). The cells were grown in a 24 well plate. 2-weeks post confluency, the cells were treated with 10 mM N-acetylcysteine (NAC) for 30 min to remove the adherent mucus layer or PBS as a control. The cells were rinsed once in PBS before inoculation with C. albicans. For infection, C. albicans strain MLR62, which constitutively expresses GFP, was diluted from an overnight culture into DMEM to OD600=0.5. 500 μL of C. albicans was added on top of the HT29-MTX cells and incubated at 37° C. for 2 hours. After 2 hours, the medium was removed from the wells which were subsequently washed with 500 μL of PBS twice. Another 500 μL of PBS was added to each well which was then read using a plate reader (ex488/em538).

### Filamentation Assay

100 μL of RPMI, 0.5% methylcellulose, 0.5% Sigma-manufactured PGM, and 0.5% native PGM were each inoculated with the strain HGFP3 as yeast-form in a 96 well plate. 1 μL of an overnight culture was inoculated into each media condition and the plates were incubated at 37° C. and 180 rpm for 8 h. Wells were scraped with a pipette tip to remove adherent cells and samples were pipetted vigorously to break up aggregates. 15 μL of each sample was placed on a microscope slide for visualization. Slides were imaged via a Zeiss Observer Z1 inverted fluorescence microscope with a Zeiss Plan-Apochromat 20× objective lens under phase contrast and FITC.

### REFERENCES FOR EXAMPLE II

1. Abramoff, M. D., P. J. Magelhaes, and S. J. Ram. 2004. Image Processing with ImageJ. Biophotonics International 11:36-42.
2. Lesuffleur, T., N. Porchet, J. P. Aubert, D. Swallow, J. R. Gum, Y. S. Kim, F. X. Real, and A. Zweibaum. 1993. Differential expression of the human mucin genes MUC1 to MUC5 in relation to growth and differentiation of different mucus-secreting HT-29 cell subpopulations. J Cell Sci 106:771-783.
3. Nobile, C. J., and A. P. Mitchell. 2005. Regulation of cell-surface genes and biofilm formation by the C. albicans transcription factor Bcrlp. Curr. Biol 15:1150-1155.
4. Staab, J. F., Y.-S. Bahn, and P. Sundstrom. 2003. Integrative, multifunctional plasmids for hypha-specific or constitutive expression of green fluorescent protein in Candida albicans. Microbiology (Reading, Engl.) 149:2977-2986.

It should be understood that for all numerical bounds describing some parameter in this application, such as “about,” “at least,” “less than,” and “more than,” the description also necessarily encompasses any range bounded by the recited values. Accordingly, for example, the description at least 1, 2, 3, 4, or 5 also describes, inter alia, the ranges 1-2, 1-3, 1-4, 1-5, 2-3, 2-4, 2-5, 3-4, 3-5, and 4-5, et cetera.

It should be understood that for all numerical bounds describing some parameter in this application, such as “about,” “at least,” “less than,” and “more than,” the description also necessarily encompasses any range bounded by the recited values. Accordingly, for example, the description at least 1, 2, 3, 4, or 5 also describes, inter alia, the ranges 1-2, 1-3, 1-4, 1-5, 2-3, 2-4, 2-5, 3-4, 3-5, and 4-5, et cetera.

For all patents, applications, or other reference cited herein, such as non-patent literature and reference sequence information, it should be understood that it is incorporated by reference in its entirety for all purposes as well as for the proposition that is recited. Where any conflict exits between a document incorporated by reference and the present application, this application will control. All information associated with reference gene sequences disclosed in this application, such as GeneIDs or accession numbers (typically referencing NCBI accession numbers), including, for example, genomic loci, genomic sequences, functional annotations, allelic variants, and reference mRNA (including, e.g., exon boundaries or response elements) and protein sequences (such as conserved domain structures) are hereby incorporated by reference in their entirety.

Headings used in this application are for convenience only and do not affect the interpretation of this application.

Preferred features of each of the aspects provided by the invention are applicable to all of the other aspects of the invention mutatis mutandis and, without limitation, are exemplified by the dependent claims and also encompass combinations and permutations of individual features (e.g. elements, including numerical ranges and exemplary embodiments) of particular embodiments and aspects of the invention including the working examples. For example, particular experimental parameters exemplified in the working examples can be adapted for use in the methods provided by the invention piecemeal without departing from the invention. For example, for materials that are disclosed or used in the methods provided by the invention, while specific reference of each various individual and collective combinations and permutation of these compounds may not be explicitly disclosed, each is specifically contemplated and described herein. Thus, if a class of elements A, B, and C are disclosed as well as a class of elements D, E, and F and an example of a combination of elements, A-D is disclosed, then even if each is not individually recited, each is individually and collectively contemplated. Thus, in this example, each of the combinations A-E, A-F, B-D, B-E, B-F, C-D, C-E, and C-F are specifically contemplated and should be considered disclosed from disclosure of A, B, and C; D, E, and F; and the example combination A-D. Likewise, any subset or combination of these is also specifically contemplated and disclosed. Thus, for example, the sub-group of A-E, B-F, and C-E are specifically contemplated and should be considered disclosed from disclosure of A, B, and C; D, E, and F; and the example combination A-D. This concept applies to all aspects of this application including, elements of a composition of matter and steps of method of making or using the compositions.

While this invention has been particularly shown and described with references to example embodiments thereof, it will be understood by those skilled in the art that various changes in form and details may be made therein without departing from the scope of the invention encompassed by the appended claims.

What is claimed is:

1. A method of inhibiting virulence of one or more microorganisms, and/or inhibiting one or more microorganisms from attaching to a surface, forming suspended aggregates or a combination thereof, comprising contacting the one or more microorganisms, the surface, or a combination thereof with purified, native, non-human mucin.

2. The method of claim 1 wherein the one or more microorganisms are planktonic microorganisms, sessile microorganisms or a combination thereof.

3. The method of claim 1 wherein the one or more microorganisms are pathogenic or capable of pathogenicity.

4. The method of claim 1 wherein the one or more microorganisms is one or more bacteria, archaea, fungi or a combination thereof.

5. The method of claim 4 wherein the one or more bacteria is an Actinobacteria, a Firmicute, a Bacteroidete, a Chlamydiae, a Fusobacteria, a Proteobacteria, a Spirochaete, or a combination thereof.

6. (canceled)

7. (canceled)

8. The method of claim 1 wherein the non-human mucin is porcine gastric mucin.

9. (canceled)

10. (canceled)

11. (canceled)

12. The method of claim 5 wherein the concentration of the mucin is about 0.20% (w/v), 0.25% (w/v), 0.30% (w/v), 0.35% (w/v), 0.40% (w/v), 0.45% (w/v), 0.50% (w/v), 0.55% (w/v), 0.6% (w/v), 0.65% (w/v), 0.7% (w/v), 0.75% (w/v), 0.8% (w/v), 0.85% (w/v), 0.9% (w/v), 0.95% (w/v), 1% (w/v), 1.5% (w/v), 2.0% (w/v), 2.5% (w/v) in the solution.

13. The method of claim 12 wherein the mucin is in a solution of phosphate buffered saline or cell culture media.

14. (canceled)

15. (canceled)

16. The method of claim 1 wherein the mucin is in a solution that has a salt concentration of about 20 mM, 40 mM, 60 mM, 80 mM, 100 mM, 120 mM, 140 mM, 160 mM, 180 mM, 200 mM, 220 mM, 240 mM, 260 mM, 280 mM, 300 mM, 320 mM, 340 mM, 360 mM, 380 mM, 400 mM, 420 mM, 440 mM, 460 mM, 480 mM, 500 mM, 520 mM, 540 mM, 560 mM, 580 mM, 600 mM, 620 mM, 640 mM, 660 mM, 680 mM, 700 mM, 720 mM, 740 mM, 760 mM, 780 mM, 800 mM, 820 mM, 840 mM, 860 mM, 880 mM, 900 mM, 920 mM, 940 mM, 960 mM, 980 mM, or 1000 mM.

17. The method of claim 1 wherein the surface is a surface of an individual, a surface of a product that is ingested by an individual, a surface of an instrument or device that comes in contact with an individual, a surface that comes in contact with water or a combination thereof.

18. A method of inhibiting one or more microorganisms from forming a biofilm comprising contacting the one or more microorganisms with purified, native non-human mucin.

19. (canceled)

20. (canceled)

21. (canceled)

22. (canceled)

23. (canceled)

24. (canceled)

25. (canceled)

26. (canceled)

27. (canceled)

28. (canceled)

29. The method of claim 18 wherein the mucin is in solution.

30. The method of claim 29 wherein the concentration of the mucin is about 0.20% (w/v), 0.25% (w/v), 0.30% (w/v), 0.35% (w/v), 0.40% (w/v), 0.45% (w/v), 0.50% (w/v), 0.55% (w/v), 0.6% (w/v), 0.65% (w/v), 0.7% (w/v), 0.75% (w/v), 0.8% (w/v), 0.85% (w/v), 0.9% (w/v), 0.95% (w/v), 1% (w/v), 1.5% (w/v), 2.0% (w/v), 2.5% (w/v) in the solution.

31. The method of claim 30 wherein the mucin is in a solution of phosphate buffered saline or cell culture media.

32. The method of claim 18 wherein the mucin is a gastric mucin that has an acidic, basic or neutral pH.

33. The method of claim 32 wherein the pH is about 2, 3, 4, 5, 6, 8, 9, 10, 11, 12, or 13.

34. The method of claim 18 wherein the mucin is in a solution that has a salt concentration of about 20 mM, 40 mM, 60 mM, 80 mM, 100 mM, 120 mM, 140 mM, 160 mM, 180 mM, 200 mM, 220 mM, 240 mM, 260 mM, 280 mM, 300 mM, 320 mM, 340 mM, 360 mM, 380 mM, 400 mM, 420 mM, 440 mM, 460 mM, 480 mM, 500 mM, 520 mM, 540 mM, 560 mM, 580 mM, 600 mM, 620 mM, 640 mM, 660 mM, 680 mM, 700 mM, 720 mM, 740 mM, 760 mM, 780 mM, 800 mM, 820 mM, 840 mM, 860 mM, 880 mM, 900 mM, 920 mM, 940 mM, 960 mM, 980 mM, or 1000 mM.

35. The method of claim 18 wherein the surface is a surface of an individual, a surface of a product that is ingested by an individual, a surface of an instrument or device that comes in contact with an individual, a surface that comes in contact with water or a combination thereof.

36. The method of claim 18, wherein the microorganism is a fungus.

37. (canceled)

38. (canceled)
